(19) United States
(12) Patent Application Publication
(10) Pub. No.: US 2016/0256365 A1
(43) Pub. Date: Sep. 8, 2016

(54) Hair Conditioning Compositions With Microcapsules

(71) Applicant: The Procter & Gamble Company, Cincinnati, OH (US)

(72) Inventors: Jiten Odhavji DIHORA, Liberty Twp, OH (US); Matthew Joseph LINSER, Cincinnati, OH (US); Matthew Benjamin TASSOS, Liberty Twp, OH (US)

(21) Appl. No.: 15/059,926

(22) Filed: Mar. 3, 2016

Related U.S. Application Data
(60) Provisional application No. 62/127,802, filed on Mar. 3, 2015.

(51) Int. Cl. A61K 8/11; A61Q 13/00; A61K 8/40; A61K 8/898; A61K 8/81; A61K 8/34; A61K 8/42; A61K 8/365; A61Q 5/12; A61K 8/37

(52) U.S. Cl. CPC A61K 8/11; A61Q 5/12; A61Q 13/00; A61K 8/40; A61K 8/37; A61K 8/8152; A61K 8/342; A61K 8/42; A61K 8/365; A61K 8/898; A61K 2800/524; A61K 2800/652; A61K 2800/592

(57) ABSTRACT

Examples and methods of providing hair conditioning compositions with microcapsules are described herein, in particular, with reference to the ratio of the encapsulated perfume oil to solute in the composition.

### TECHNICAL FIELD

The present disclosure relates to hair conditioning compositions that provide blooms of fragrances through the use of microcapsules.

### BACKGROUND

Consumers often desire consumer products for the many benefits they may provide. For example, it is not uncommon for a particular consumer to have in their home shampoos, conditioners, body washes, deodorants, fine fragrances, shaving gels, etc. Often, such consumer products also include fragrances. Such fragrances may delight the user by providing a freshness feeling and may serve as a signal to the user that the product may still be working or that the product is still present. Yet because of the volatility of many fragrances and/or habituation, a consumer may be unable to notice the fragrance shortly after using/applying the consumer product, potentially leading the user to believe the benefits are dissipating or have dissipated. Consequentially, it may be desirable to have technologies than improve the noticeability of fragrances in consumer products.

### SUMMARY

A method for treating hair, the method comprising: providing a hair conditioner composition, the composition comprising a solute and a carrier, said solute comprising a conditioning agent and a plurality of microcapsules, wherein said microcapsules comprise an encapsulated perfume oil, wherein the weight ratio of the encapsulated perfume oil to the solute is greater than about 0.02; and applying from about 0.01 grams to about 3.4 gram per gram of hair or less per use.

### BRIEF DESCRIPTION OF THE DRAWINGS

While the specification concludes with claims, it is believed that the same will be better understood from the following description taken in conjunction with the accompanying drawings in which:

FIG. 1 is a graph illustrating the Primavera Grade of perfume released into the headspace by leave-on conditioners containing microcapsules that vary in the ratio of the perfume to solute ratios and that were applied to hair switches at different doses and combed 24 hours after application to the hair switches.

FIG. 2 is a graph illustrating the Primavera Grade of perfume released into the headspace by leave-on conditioners containing microcapsules that vary percent of solute and that were applied to hair switches at different doses and combed 24 hours after application to the hair switches.

### DETAILED DESCRIPTION

Components of the hair conditioning compositions (e.g., leave-on conditioners) are described below. Also included is a nonexclusive description of various optional and preferred components useful in embodiments of the present invention. While the specification concludes with claims that particularly point out and distinctly claim the invention, it is believed the present invention will be better understood from the following description.

All percentages, parts, and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The compositions and methods/processes of the present invention can comprise, consist of, and consist essentially of the essential elements and limitations of the invention described herein, as well as any of the additional or optional ingredients, components, steps, or limitations described herein.

“Effective amount” means an amount sufficient enough to provide a dry conditioning benefit.

“Mixtures” means a combination of materials in any combination.

“Molecular weight” or “M.Wt.” as used herein refers to the weight average molecular weight unless otherwise stated.

“pH QS” means the amount required to adjust the pH accordingly.

“PMC” means a microcapsule having a shell and a core and wherein the core includes at least one perfume oil.

“QS” means the amount of material required to bring the total to 100%.

“Solute” refers to all of the material in a composition excluding the carrier(s).

“Substantially free of” means an amount of a material that is less than 1%, 0.5%, 0.25%, 0.1%, 0.05%, 0.01%, or 0.001% by weight of a composition.

“Visc. QS” means the amount of material required to adjust the viscosity accordingly.

Examples of the hair conditioning compositions may include silicone polymers having a viscosity of up to 100,000 mPa·s.

The hair conditioning compositions herein may have a pH of from about 2 to about 9, preferably from about 3 to about 7.

### Introduction

The hair conditioning compositions and treatments described herein may deliver consistent blooms of fragrance from the microcapsules. It has been surprisingly discovered that the amount applied of a leave-on composition and the ratio of microcapsules to the solute within the leave-on composition may impact the performance of the microcapsules when applied to human hair. In this regard, it has been discovered that when the ratio of solute to perfume within the microcapsules is controlled, users of the leave-on compositions can experience a consistent experience from the PMCs, irrespective of the dose applied to the hair. When the ratio of the solute to the perfume within the microcapsules is not controlled, then the user may observe a reduction in the performance of the microcapsules despite applying a greater amount of the leave-on composition onto the hair. By understanding the relationship of the solute to the microcapsules, leave-on compositions and treatments can be generated that maximize the performance of the microcapsules while also minimizing the amount of microcapsules required.

Typically, a formulator would expect that increasing the amount of perfume in the composition will increase the perceptibility of the perfume. Table A represents such an expectation. Example 3A represents a leave-on conditioner with 0.3%, by weight of the microcapsules of Example 2, Example 3B represents a leave-on conditioner with 0.6% by weight of the microcapsules of Example 2, and Example 3C represents a leave-on conditioner with 1.0% by weight of the microcapsules of Example 2. Example 3D represents a leave-on conditioner with 0.3% by weight of the microcapsules of Example 1, Example 3E represents a leave-on conditioner with 0.6% by weight of the microcapsules of Example 1, and Example 3F represents a leave-on conditioner with 1.0% by weight of the microcapsules of Example 1. For all leave-on conditioner examples tested, the encapsulated perfume is the only fragrance added to the compositions. Example 3J represents a leave-on conditioner with 0.3% by weight of the microcapsules of Example 2, Example 3K represents a leave-on conditioner with 0.6% by weight of the microcapsules of Example 2, and Example 3L represents a leave-on conditioner with 1.0% by weight of the microcapsules of Example 2. For leave-on conditioner Example 3J, Example 3K, and Example 3L, 0.4% of a neat fragrance is added to the compositions.

Examples 3A-3L are used to prepare hair switches per Hair Switch Treatment method, and allowed to dry for 4 hours. Next, the Olfactive Analysis Method is utilized to gather perfume intensity data on the prepared hair switches before and after combing. The hair switches are combed a second time, then the Olfactive Analysis Method is utilized, with the exception that no perfume intensity data is obtained. The hair switches are allowed to age for 24 hours or 48 hours and then the same procedure is followed to obtain perfume intensity data.

As illustrated in Table A, as the percentage of perfume within the leave-on conditioner increases, so does the performance. Comparing Example 3A to Example 3C, increasing the amount of perfume in the composition from 0.3% (Example 3A) to 1% (Example 3C) resulted in an increase in the olfactive grade at 24 hr from 40 to 50, respectively. Comparing Example 3D to Example 3F, increasing the amount of perfume in the composition from 0.3% (Example 3D) to 1% (Example 3F) resulted in an increase in the olfactive grade at 24 hr from 50 to 55, respectively. Comparing Example 3J to Example 3L, increasing the amount of perfume by way of microcapsules in the composition from 0.3% (Example 3J) to 1% (Example 3L) resulted in an increase in the olfactive grade at 24 hr from 40 to 50, respectively. These results demonstrate what one of ordinary skill in the art would expect—a higher olfactive grade should be observed as one increases the percentage of microcapsules containing a perfume oil in a leave-on conditioner.

TABLE A

| | Weight % of | | Olfactive Grade | Olfactive Grade |
|---|---|---|---|---|
| | Perfume (by way | Quantity of | at first comb, | at third comb, |
| | of the | Product Applied | 4 hr post- | 24 hr post- |
| | microcapsules) | to hair | combing | combing |
| Example 3A | 0.3% | 1 g | 40 | 40 |
| | | (0.1 g product/g | | |
| | | of hair) | | |
| Example 3B | 0.6% | 1 g | 50 | 45 |
| | | (0.1 g product/g | | |
| | | of hair) | | |
| Example 3C | 1.0% | 1 g | 50 | 50 |
| | | (0.1 g product/g | | |
| | | of hair) | | |
| Example 3D | 0.3% | 0.4 g | 50 | 50 |
| | | (0.1 g product/g | | |
| | | of hair) | | |
| Example 3E | 0.6% | 0.4 g | 55 | 50 |
| | | (0.1 g product/g | | |
| | | of hair) | | |
| Example 3F | 1.0% | 0.4 g | 60 | 55 |
| | | (0.1 g product/g | | |
| | | of hair) | | |
| Example 3J | 0.3% | 0.4 g | 55 | 40 |
| | | (0.1 g product/g | | |
| | | of hair) | | |
| Example 3K | 0.6% | 0.4 g | 60 | 45 |
| | | (0.1 g product/g | | |
| | | of hair) | | |
| Example 3L | 1.0% | 0.4 g | 65 | 50 |
| | | (0.1 g product/g | | |
| | | of hair) | | |

However, it has been surprisingly found that the amount of leave-on conditioner applied to the hair may impact the performance of the PMCs. In this regard, different amounts of Examples 3A, 3D and 3J were applied to a consistent amount of hair and the olfactive grade was measured 24 and 48 hrs after application. As shown in Table B, increasing the dose of Example 3A from 1 gram of product to 3.33 grams of product decreased the olfactive grade of the product at 48 hr from 35 to 25. Similarly, increasing the dose of Example 3D from 0.4 grams of product to 1.33 grams of product decreased the olfactive grade of the product at 48 hr from 47.5 to 32.5. Similarly, increasing the dose of Example 3J from 0.4 grams of product to 1.33 grams of product decreased the olfactive grade of the product at 48 hr from 45 to 35. These results suggest that the dose of a leave-on conditioner containing PMCs may be an important variable in achieving maximum/noticeable performance from the microcapsules.

TABLE B

| | | Quantity | | Weight Ratio | Olfactive | Olfactive |
|---|---|---|---|---|---|---|
| | Quantity | of Perfume (by | | of Perfume (by | Grade | Grade |
| | of Product | way of the | Quantity | way of the | at third comb, | at fifth comb, |
| | Applied | microcapsules) | of Solute | microcapsules) | 24 hr post- | 48 hr post- |
| | to Hair | on Hair | on Hair | to Solute | combing | combing |
| Example | 1 g | 0.003 g | 0.105 g | 0.028 | 40 | 35 |
| 3A | (0.1 g product/ | | | | | |
| | g of hair) | | | | | |
| Example | 2 g | 0.006 g | 0.211 g | 0.028 | 30 | 30 |
| 3A | (0.2 g product/ | | | | | |
| | g of hair) | | | | | |
| Example | 3.33 g | 0.010 g | 0.351 g | 0.028 | 25 | 25 |
| 3A | (0.333 g product/ | | | | | |
| | g of hair) | | | | | |
| Example | 0.4 g | 0.0012 g | 0.0422 g | 0.028 | 50 | 47.5 |
| 3D | (0.1 g product/ | | | | | |
| | g of hair) | | | | | |
| Example | 0.80 g | 0.0024 g | 0.0843 g | 0.028 | 50 | 37.5 |
| 3D | (0.2 g product/ | | | | | |
| | g of hair) | | | | | |
| Example | 1.33 g | 0.0040 g | 0.140 g | 0.028 | 42.5 | 32.5 |
| 3D | (0.333 g product/ | | | | | |
| | g of hair) | | | | | |
| Example | 0.4 g | 0.0012 g | 0.0422 g | 0.028 | 40 | 45 |
| 3J | (0.1 g product/ | | | | | |
| | g of hair) | | | | | |
| Example | 0.8 g | 0.0024 g | 0.0843 g | 0.028 | 40 | 30 |
| 3J | (0.2 g product/ | | | | | |
| | g of hair) | | | | | |
| Example | 1.33 g | 0.0040 g | 0.140 g | 0.028 | 35 | 35 |
| 3J | (0.333 g product/ | | | | | |
| | g of hair) | | | | | |

To determine whether the amount of perfume was a factor in driving the negative performance as the dose of leave-on conditioner is increased, Example 4A/3C and 4B/3A were formulated so that 0.010 g of perfume are delivered via the application of 1 gram or 3 grams of product, respectively. Example 4A and 4B also contain a weight ratio of perfume to solute of 0.272 and 0.115, respectively. As shown in Table C, the application of 1 gram of Example 4A resulted in an olfactive grade of 67.5 at 24 hr and 62.5 at 48 hr. In contrast, the application of 3.33 grams of Example 4B resulted in an olfactive grade of 60 at 24 hr and 52.5 at 48 hr. Similarly, the application of 1 gram of Example 3C resulted in an olfactive grade of 50 at 24 hr and 40 at 48 hr while the application of 3.33 grams of Example 3A resulted in an olfactive grade of 25 at 24 hr and 25 at 48 hr. Similarly, Example 3F/3L and 3D/3J were formulated so that 0.0040 grams of perfume are delivered via the application of 0.4 grams or 1.33 grams of product, respectively. Example 3F and 3D also contain a weight ratio of perfume (by way of the microcapsules) to solute of 0.086 and 0.028, respectively. As shown in Table C, the application of 0.4 grams of Example 3F resulted in an olfactive grade of 55 at 24 hr and 47.5 at 48 hr. In contrast, the application of 1.33 grams of Example 3D resulted in an olfactive grade of 42.5 at 24 hr and 32.5 at 48 hr. Similarly, the application of 0.4 grams of Example 3L resulted in an olfactive grade of 50 at 24 hr and 45 at 48 hr while the application of 1.33 grams of Example 3J resulted in an olfactive grade of 35 at 24 hr and 35 at 48 hr. These results suggest that higher doses of a leave-on conditioner may decrease the performance of the microcapsules even when the amount of perfume by way of the microcapsules is normalized per dose. These results also suggest that the weight ratio of the solute to perfume may be an important factor in maximizing the performance of the microcapsules in a leave-on conditioner.

TABLE C

| | | Quantity | | Weight Ratio | Olfactive | Olfactive |
|---|---|---|---|---|---|---|
| | Quantity | of Perfume | | of Perfume (by | Grade | Grade |
| | of Product | on Hair (by | Quantity | way of the | at third comb, | at fifth comb, |
| | Applied | way of the | of Solute | microcapsules) | 24 hr post- | 48 hr post- |
| | to hair | microcapsules) | on Hair | to Solute | combing | combing |
| Example | 1 gram | 0.010 g | 0.0367 g | 0.272 | 67.5 | 62.5 |
| 4A | (0.1 g product/ | | | | | |
| | g of hair) | | | | | |
| Example | 3.33 grams | 0.010 g | 0.0869 g | 0.115 | 60 | 52.5 |
| 4B | (0.333 g product/ | | | | | |
| | g of hair) | | | | | |
| Example | 1.0 grams | 0.010 g | 0.116 g | 0.086 | 50 | 40 |
| 3C | (0.1 g product/ | | | | | |
| | g of hair) | | | | | |
| Example | 3.33 grams | 0.010 g | 0.351 g | 0.028 | 25 | 25 |
| 3A | (0.333 g product/ | | | | | |
| | g of hair) | | | | | |
| Example | 0.4 g | 0.0040 g | 0.0464 g | 0.086 | 55 | 47.5 |
| 3F | (0.1 g product/ | | | | | |
| | g of hair) | | | | | |
| Example | 1.33 g | 0.0040 g | 0.140 g | 0.028 | 42.5 | 32.5 |
| 3D | (0.333 g product/ | | | | | |
| | g of hair) | | | | | |
| Example | 0.4 g | 0.0040 g | 0.0464 g | 0.086 | 50 | 45 |
| 3L | (0.1 g product/ | | | | | |
| | g of hair) | | | | | |
| Example | 1.33 g | 0.0040 g | 0.140 g | 0.028 | 35 | 35 |
| 3J | (0.333 g product/ | | | | | |
| | g of hair) | | | | | |

A leave-on conditioner containing PMCs was then formulated so that the weight ratio of perfume delivered via the microcapsules to total solute delivered is controlled. Without being limited to theory, it is believed that the weight ratio of perfume delivered (via the microcapsules) to the total solute delivered to the hair/scalp is an important consideration in order to maximize the performance of the microcapsules, irrespective of the dose applied. In this regard, by achieving a favorable weight ratio of perfume (via microcapsules) to total solute, a leave-on conditioner may be formulated such that the performance of the microcapsules is not largely affected by the dose used. This can be an important feature for a leave-on conditioner that contains PMCs as the amount applied by users may vary, and it may be desired to give the user dosing flexibility to dial-in the desired experience.

As shown in Table D, when the weight ratio of perfume delivered (via the microcapsules) to the total solute delivered to the hair/scalp is favorable, varying doses of leave-on conditioner may be applied to the hair without largely affecting the performance of the microcapsules. In this regard, Example 3G, 3H, and 3I were formulated such that all three examples were at a weight ratio of perfume (via the microcapsules) to solute of 0.086. Examples 3G, 3H, and 3I were also formulated so that a consistent amount of perfume was delivered despite the variations in the dose applied to the hair. Comparing Example 3G to 3I, delivering 0.8 grams of Example 3G led to an olfactive grade at 24 hr of 50 while delivering 0.24 grams of Example 3I led to an olfactive grade at 24 hr of 45. These results suggest that when the weight ratio of perfume delivered (via the microcapsules) to the total solute delivered to the hair/scalp is favorable, varying doses of the leave-on conditioner may be applied without largely impacting the performance of the microcapsules.

TABLE D

| | | | | | Olfactive |
|---|---|---|---|---|---|
| | | | | | Grade |
| | Quantity of | Quantity of | Quantity of | Weight Ratio | at third comb, |
| | Product Applied | Perfume on | Solute on | of Perfume to | 24 hr post- |
| | to hair | Hair | Hair | Solute | combing |
| Example | 0.8 gram | 0.0024 g | 0.0278 g | 0.086 | 50 |
| 3G | (0.2 g product/g | | | | |
| | hair) | | | | |
| Example | 0.4 grams | 0.0024 g | 0.0278 g | 0.086 | 45 |
| 3H | (0.1 g product/g | | | | |
| | hair) | | | | |
| Example | 0.24 grams | 0.0024 g | 0.0278 g | 0.086 | 45 |
| 3I | (0.06 g product/ | | | | |
| | g hair) | | | | |

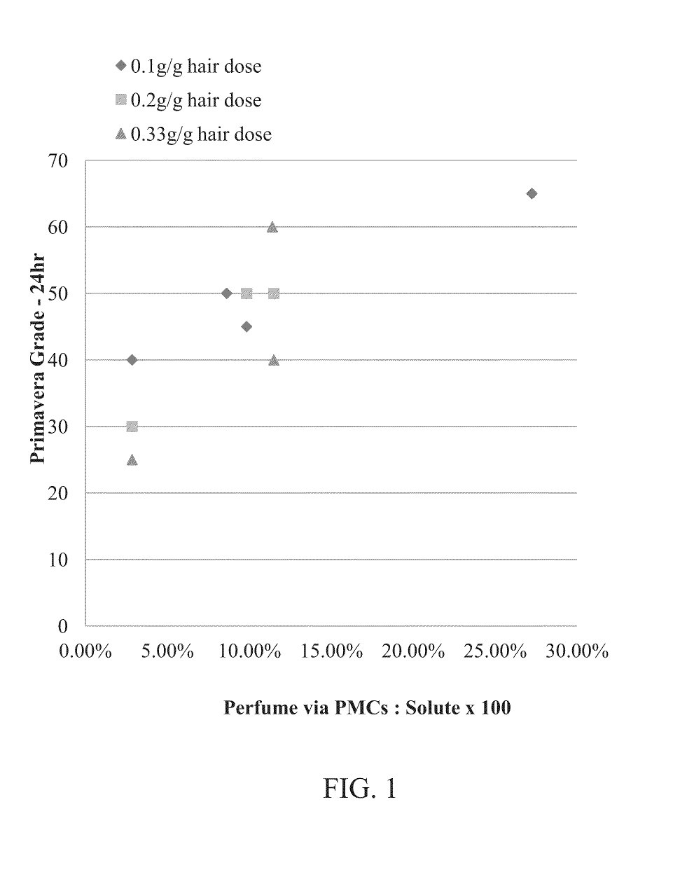

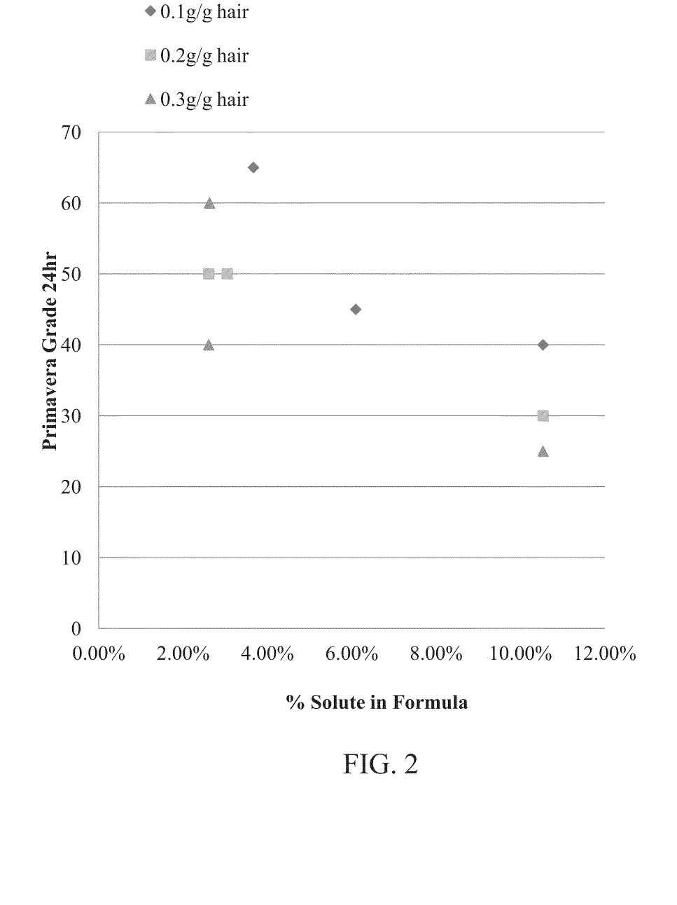

It is believed that when the weight ratio of the perfume (via the PMCs) to the solute in the leave-on conditioner is less than about 0.02, then the dose of the leave-on conditioner should not exceed about 0.1 g/g of hair in order to achieve a consumer noticeable benefit (See FIGS. 1-2). Under the Olfactive Analysis Method disclosed herein, a score of 35 or higher typically signifies a consumer noticeable benefit. When the weight ratio of the perfume (via the PMCs) to the solute in the leave-on conditioner is greater than about 0.02, then the dose of the leave-on conditioner may exceed 0.1 g/g of hair in order to achieve a consumer noticeable benefit. In some examples, such a leave-on conditioner may be applied at a dose from about 0.1 g/g of hair to about 0.34 g/g of hair. In general, the higher the weight ratio of the perfume (via the PMCs) to the solute in the leave-on conditioner, the more likely a consumer will notice the benefit from the PMCs. In some examples, significantly increasing the dosing beyond 0.333 grams per gram of hair may result in dosing levels where the consumer may feel negative about the product. Some of these negative attributes include too much product weighing down of the hair, excessive product that drips and creates a mess, longer drying times required, or a reduction in the performance of the PMCs. In some examples, the weight ratio of the encapsulated perfume oil to solute is from about 0.02 to about 0.7, alternatively from about 0.1 to about 0.5.

Microcapsules/Perfume Oils

The microcapsules may be any kind of microcapsule disclosed herein or known in the art. The microcapsules may be included from about 0.01% to about 45%, by weight, of the composition. The microcapsules may have a shell and a core material encapsulated by the shell. The core material of the microcapsules may include one or more perfume oils. The shells of the microcapsules may be made from synthetic polymeric materials or naturally-occurring polymers. Synthetic polymers may be derived from petroleum oil, for example. Non-limiting examples of synthetic polymers include nylon, polyethylenes, polyamides, polystyrenes, polyisoprenes, polycarbonates, polyesters, polyureas, polyurethanes, polyolefins, polysaccharides, epoxy resins, vinyl polymers, polyacrylates, and mixtures thereof. Natural polymers occur in nature and may often be extracted from natural materials. Non-limiting examples of naturally occurring polymers are silk, wool, gelatin, cellulose, proteins, and combinations thereof.

The microcapsules may be friable microcapsules. A friable microcapsule is configured to release its core material when its shell is ruptured. The rupture may be caused by forces applied to the shell during mechanical interactions. The microcapsules may have a shell with a volume weighted fracture strength of from about 0.1 mega Pascals to about 15.0 mega Pascals, when measured according to the Fracture Strength Test Method described herein, or any incremental value expressed in 0.1 mega Pascals in this range, or any range formed by any of these values for fracture strength. As an example, a microcapsule may have a shell with a volume weighted fracture strength of 0.8-15.0 mega Pascals (MPa), alternatively from 5.0-12.0 mega Pascals (MPa), or alternatively from 6.0-10.0 mega Pascals (MPa).

The microcapsules may have a median volume-weighted particle size of from 2 microns to 80 microns, from 10 microns to 30 microns, or from 10 microns to 20 microns, as determined by the Test Method for Determining Median Volume-Weighted Particle Size of Microcapsules described herein.

The microcapsules may have various core material to shell weight ratios. The microcapsules may have a core material to shell ratio that is greater than or equal to: 70% to 30%, 75% to 25%, 80% to 20%, 85% to 15%, 90% to 10%, and 95% to 5%.

The microcapsules may have shells made from any material in any size, shape, and configuration known in the art. Some or all of the shells may include a polyacrylate material, such as a polyacrylate random copolymer. For example, the polyacrylate random copolymer may have a total polyacrylate mass, which includes ingredients selected from the group including: amine content of 0.2-2.0% of total polyacrylate mass; carboxylic acid of 0.6-6.0% of total polyacrylate mass; and a combination of amine content of 0.1-1.0% and carboxylic acid of 0.3-3.0% of total polyacrylate mass.

When a microcapsule's shell includes a polyacrylate material, and the shell has an overall mass, the polyacrylate material may form 5-100% of the overall mass, or any integer value for percentage in this range, or any range formed by any of these values for percentage. As examples, the polyacrylate material may form at least 5%, at least 10%, at least 25%, at least 33%, at least 50%, at least 70%, or at least 90% of the overall mass.

Some or all of the microcapsules may have various shell thicknesses. For at least a first group of the provided microcapsules, each microcapsule may have a shell with an overall thickness of 1-300 nanometers, or any integer value for nanometers in this range, or any range formed by any of these values for thickness. As an example, microcapsules may have a shell with an overall thickness of 2-200 nanometers.

The microcapsules may also encapsulate one or more benefit agents. The benefit agent(s) include, but are not limited to, cooling sensates, warming sensates, perfume oils, oils, pigments, dyes, chromogens, phase change materials, and other kinds of benefit agent known in the art, in any combination. In some examples, the perfume oil encapsulated may have a C log P of less than 4.5 or a C log P of less than 4. Alternatively the perfume oil encapsulated may have a C log P of less than 3. In some examples, the microcapsule may be anionic, cationic, zwitterionic, or have a neutral charge. The benefit agents(s) may be in the form of solids and/or liquids. The benefit agent(s) may be any kind of perfume oil(s) known in the art, in any combination.

The microcapsules may encapsulate a partitioning modifier in addition to the benefit agent. Non-limiting examples of partitioning modifiers include isopropyl myristate, mono-, di-, and tri-esters of C₄-C₂₄ fatty acids, castor oil, mineral oil, soybean oil, hexadecanoic acid, methyl ester isododecane, isoparaffin oil, polydimethylsiloxane, brominated vegetable oil, and combinations thereof. Microcapsules may also have varying ratios of the partitioning modifier to the benefit agent so as to make different populations of microcapsules that may have different bloom patterns. Such populations may also incorporate different perfume oils so as to make populations of microcapsules that display different bloom patterns and different scent experiences. U.S. 2011-0268802 discloses other non-limiting examples of microcapsules and partitioning modifiers and is hereby incorporated by reference.

The microcapsule's shell may comprise a reaction product of a first mixture in the presence of a second mixture comprising an emulsifier, the first mixture comprising a reaction product of i) an oil soluble or dispersible amine with ii) a multifunctional acrylate or methacrylate monomer or oligomer, an oil soluble acid and an initiator, the emulsifier comprising a water soluble or water dispersible acrylic acid alkyl acid copolymer, an alkali or alkali salt, and optionally a water phase initiator. In some examples, said amine is an aminoalkyl acrylate or aminoalkyl methacrylate.

The microcapsules may include a core material and a shell surrounding the core material, wherein the shell comprises: a plurality of amine monomers selected from the group consisting of aminoalkyl acrylates, alkyl aminoalkyl acrylates, dialkyl aminoalykl acrylates, aminoalkyl methacrylates, alkylamino aminoalkyl methacrylates, dialkyl aminoalykl methacrylates, tertiarybutyl aminethyl methacrylates, diethylaminoethyl methacrylates, dimethylaminoethyl methacrylates, dipropylaminoethyl methacrylates, and mixtures thereof; and a plurality of multifunctional monomers or multifunctional oligomers. Non-limiting examples of emulsifiers include water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, gum arabic, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, phosphated or sulfated tristyrylphenol ethoxylates, palmitamidopropyltrimonium chloride (Varisoft PATC™, available from Degussa Evonik, Essen, Germany), distearyl dimonium chloride, cetyltrimethylammonium chloride, quaternary ammonium compounds, fatty amines, aliphatic ammonium halides, alkyldimethylbenzylammonium halides, alkyldimethylethylammonium halides, polyethyleneimine, poly(2-dimethylamino)ethyl methacrylate) methyl chloride quaternary salt, poly(l-vinylpyrrolidone-co-2-dimethylaminoethyl methacrylate), poly(acrylamide-co-diallyldimethylammonium chloride), poly(allylamine), poly[bis(2-chloroethyl) ether-all-1,3-bis[3-(dimethylamino)propyl]urea] quaternized, and poly(dimethylamine-co-epichlorohydrin-co-ethylenediamine), condensation products of aliphatic amines with alkylene oxide, quaternary ammonium compounds with a long-chain aliphatic radical, e.g. distearyldiammonium chloride, and fatty amines, alkyldimethylbenzylammonium halides, alkyldimethylethylammonium halides, polyalkylene glycol ether, condensation products of alkyl phenols, aliphatic alcohols, or fatty acids with alkylene oxide, ethoxylated alkyl phenols, ethoxylated arylphenols, ethoxylated polyaryl phenols, carboxylic esters solubilized with a polyol, polyvinyl alcohol, polyvinyl acetate, or copolymers of polyvinyl alcohol polyvinyl acetate, polyacrylamide, poly(N-isopropylacrylamide), poly(-hydroxypropyl methacrylate), poly(-ethyl-2-oxazoline), poly(2-isopropenyl-2-oxazoline-co-methyl methacrylate), poly(methyl vinyl ether), and polyvinyl alcohol-co-ethylene), and cocoamidopropyl betaine.

Process for making microcapsules are well known. Various processes for microencapsulation, and exemplary methods and materials, are set forth in U.S. Pat. No. 6,592,990; U.S. Pat. No. 2,730,456; U.S. Pat. No. 2,800,457; U.S. Pat. No. 2,800,458; U.S. Pat. No. 4,552,811; and U.S. 2006/0263518 A1.

The microcapsule may be spray-dried to form spray-dried microcapsules. The composition may also contain one or more additional delivery systems for providing one or more benefit agents, in addition to the microcapsules. The additional delivery system(s) may differ in kind from the microcapsules. For example, wherein the microcapsule encapsulates a perfume oil, the additional delivery system may be an additional fragrance delivery system, such as a moisture-triggered fragrance delivery system. Non-limiting examples of moisture-triggered fragrance delivery systems include cyclic oligosaccharide, starch (or other polysaccharide material), starch derivatives, and combinations thereof. Said polysaccharide material may or may not be modified.

The plurality of microcapsules may include anionic, cationic, and non-ionic microcapsules, in any combination, when included in a composition with a pH range of from 2 to about 10, alternatively from about 3 to about 9, alternatively from about 4 to about 8.

In some examples, the microcapsules may include a benefit agent comprising: a.) a perfume composition having a C log P of less than 4.5; b.) a perfume composition comprising, based on total perfume composition weight, 60% perfume materials having a C log P of less than 4.0; c.) a perfume composition comprising, based on total perfume composition weight, 35% perfume materials having a C log P of less than 3.5; d.) a perfume composition comprising, based on total perfume composition weight, 40% perfume materials having a C log P of less than 4.0 and at least 1% perfume materials having a C log P of less than 2.0; e.) a perfume composition comprising, based on total perfume composition weight, 40% perfume materials having a C log P of less than 4.0 and at least 15% perfume materials having a C log P of less than 3.0; f.) a perfume composition comprising, based on total perfume composition weight, at least 1% butanoate esters and at least 1% of pentanoate esters; g.) a perfume composition comprising, based on total perfume composition weight, at least 2% of an ester comprising an allyl moiety and at least 10% of another perfume comprising an ester moiety; h.) a perfume composition comprising, based on total perfume composition weight, at least 1% of an aldehyde comprising an alkyl chain moiety; i.) a perfume composition comprising, based on total perfume composition weight, at least 2% of a butanoate ester; j.) a perfume composition comprising, based on total perfume composition weight, at least 1% of a pentanoate ester; k.) a perfume composition comprising, based on total perfume composition weight, at least 3% of an ester comprising an allyl moiety and 1% of an aldehyde comprising an alkyl chain moiety; a perfume composition comprising, based on total perfume composition weight, at least 25% of a perfume comprising an ester moiety and 1% of an aldehyde comprising an alkyl chain moiety; m.) a perfume compositions comprising, based on total perfume composition weight, at least 2% of a material selected from 4-(2,6,6-trimethyl-1-cyclohexenyl)-3-buten-2-one, 4-(2,6,6-trimethyl-2-cyclohexenyl)-3-buten-2-one and 3-buten-2-one,3-methyl-4-(2,6,6-trimethyl-1-cyclohexen-2-yl)- and mixtures thereof; n.) a perfume composition comprising, based on total perfume composition weight, at least 0.1% of tridec-2-enonitrile, and mandaril, and mixtures thereof; o.) a perfume composition comprising, based on total perfume composition weight, at least 2% of a material selected from 3,7-dimethyl-6-octene nitrile, 2-cyclohexylidene-2-phenylacetonitrile and mixtures thereof; p.) a perfume composition comprising, based on total perfume composition weight, at least 80% of one or more perfumes comprising a moiety selected from the group consisting of esters, aldehydes, ionones, nitriles, ketones and combinations thereof; q.) a perfume composition comprising, based on total perfume composition weight, at least 3% of an ester comprising an allyl moiety; a perfume composition comprising, based on total perfume composition weight, at least 20% of a material selected from the group consisting of: 1-methylethyl-2-methylbutanoate; ethyl-2-methyl pentanoate; 1,5-dimethyl-1-ethenylhexyl-4-enyl acetate; p-metnh-1-en-8-yl acetate; 4-(2,6,6-trimethyl-2-cyclohexenyl)-3-buten-2-one; 4-acetoxy-3-methoxy-1-propenylbenzene; 2-propenyl cyclohexanepropionate; bicyclo[2.2.1]hept-5-ene-2-carboxylic acid,3-(1-methylethyl)-ethyl ester; bycyclo[2.2.1]heptan-2-ol, 1,7,7-trimethyl-, acetate; 1,5-dimethyl-1-ethenylhex-4-enylacetate; hexyl 2-methyl propanoate; ethyl-2-methylbutanoate; 4-undecanone; 5-heptyldihydro-2(3h)-furanone; 1,6-nonadien-3-ol,3,7dimethyl-; 3,7-dimethylocta-1,6-dien-3-o; 3-cyclohexene-1-carboxaldehyde,dimethyl-; 3,7-dimethyl-6-octene nitrile; 4-(2,6,6-trimethyl-1-cyclohexenyl)-3-buten-2-one; tridec-2-enonitrile; patchouli oil; ethyl tricycle [5.2.1.0]decan-2-carboxylate; 2,2-dimethyl-cyclohexanepropanol; hexyl ethanoate, 7-acetyl,1,2,3,4,5,6,7,8-octahydro-1,1,6,7-tetramethyl naphtalene; allyl-cyclohexyloxy acetate; methyl nonyl acetic aldehyde; 1-spiro[4,5]dec-7-en-7-yl-4-pentenen-1-one; 7-octen-2-ol,2-methyl-6-methylene-,dihydro; cyclohexanol,2-(1,1-dimethylethyl)-, acetate; hexahydro-4,7-methanoinden-5(6)-yl propionatehexahydro-4,7-methanoinden-5(6)-yl propionate; 2-methoxynaphtalene; 1-(2,6,6-trimethyl-3-cyclohexenyl)-2-buten-1-one; 1-(2,6,6-trimethyl-2-cyclohexenyl)-2-buten-1-one; 3,7-dimethyloctan-3-ol; 3-buten-2-one,3-methyl-4-(2,6,6-trimethyl-1-cyclohexen-2-yl)-; hexanoic acid, 2-propenyl ester; (z)-non-6-en-1-al; 1-decyl aldehyde; 1-octanal; 4-t-butyl-α-methylhydrocinnamaldehyde; alpha-hexylcinnamaldehyde; ethyl-2,4-hexadienoate; 2-propenyl 3-cyclohexanepropanoate; and mixtures thereof; r.) a perfume composition comprising, based on total perfume composition weight, at least 20% of a material selected from the group consisting of: 1-methylethyl-2-methylbutanoate; ethyl-2-methyl pentanoate; 1,5-dimethyl-1-ethenylhex-4-enyl acetate; p-menth-1-en-8-yl acetate; 4-(2,6,6-trimethyl-2-cyclohexenyl)-3-buten-2-one; 4-acetoxy-3-methoxy-1-propenylbenzene; 2-propenyl cyclohexanepropionate; bicyclo[2.2.1]hept-5-ene-2-carboxylic acid, 3-(1-methylethyl)-ethyl ester; bycyclo[2.2.1]heptan-2-ol, 1,7,7-trimethyl-, acetate; 1,5-dimethyl-1-ethenylhex-4-enyl acetate; hexyl 2-methyl propanoate; ethyl-2-methylbutanoate,4-undecanolide; 5-heptyldihydro-2(3h)-furanone; 5-hydroxydodecanoic acid; decalactones; undecalactones, 1,6-nonadien-3-ol,3,7dimethyl-; 3,7-dimethylocta-1,6-dien-3-ol; 3-cyclohexene-1-carboxaldehyde,dimethyl-; 3,7-dimethyl-6-octene nitrile; 4-(2,6,6-trimethyl-1-cyclohexenyl)-3-buten-2-one; tridec-2-enonitrile; patchouli oil; ethyl tricycle [5.2.1.0]decan-2-carboxylate; 2,2-dimethyl-cyclohexanepropanol; allyl-cyclohexyloxy acetate; methyl nonyl acetic aldehyde; 1-spiro[4,5]dec-7-en-7-yl-4-pentenen-1-one; 7-octen-2-ol,2-methyl-6-methylene-,dihydro, cyclohexanol,2-(1,1-dimethylethyl)-, acetate; hexahydro-4,7-methanoinden-5(6)-yl propionatehexahydro-4,7-methanoinden-5(6)-yl propionate; 2-methoxynaphtalene; 1-(2,6,6-trimethyl-3-cyclohexenyl)-2-buten-1-one; 1-(2,6,6-trimethyl-2-cyclohexenyl)-2-buten-1-one; 3,7-dimethyloctan-3-ol; 3-buten-2-one,3-methyl-4-(2,6,6-trimehtyl-1-cyclohexen-2-yl)-; hexanoic acid, 2-propenyl ester; (z)-non-6-en-1-al; 1-decyl aldehyde; 1-octanal; 4-t-butyl-α-methylhydrocinnamaldehyde; ethyl-2,4-hexadienoate; 2-propenyl 3-cyclohexanepropanoate; and mixtures thereof; s.) a perfume composition comprising, based on total perfume composition weight, at least 5% of a material selected from the group consisting of 3-cyclohexene-1-carboxaldehyde,dimethyl-; 3-buten-2-one,3-methyl-4-(2,6,6-trimehtyl-1-cyclohexen-2-yl)-; patchouli oil; Hexanoic acid, 2-propenyl ester; 1-Octanal; 1-decyl aldehyde; (z)-non-6-en-1-al; methyl nonyl acetic aldehyde; ethyl-2-methylbutanoate; 1-methylethyl-2-methylbutanoate; ethyl-2-methyl pentanoate; 4-hydroxy-3-ethoxybenzaldehyde; 4-hydroxy-3-methoxybenzaldehyde; 3-hydroxy-2-methyl-4-pyrone; 3-hydroxy-2-ethyl-4-pyrone and mixtures thereof; t.) a perfume composition comprising, based on total perfume composition weight, less than 10% perfumes having a C log P greater than 5.0; u.) a perfume composition comprising geranyl palmitate; or v.) a perfume composition comprising a first and an optional second material, said first material having: (i) a C log P of at least 2; (ii) a boiling point of less than about 280° C.; and second optional second material, when present, having (i) a C log P of less than 2.5; and (ii) a ODT of less than about 100 ppb.

In some examples, the microcapsules may include a benefit agent comprising: one or more materials selected from the group consisting of (5-methyl-2-propan-2-ylcyclohexyl) acetate; 3,7-dimethyloct-6-en-1-al; 2-(phenoxy)ethyl 2-methylpropanoate; prop-2-enyl 2-(3-methylbutoxy)acetate; 3-methyl-1-isobutylbutyl acetate; prop-2-enyl hexanoate; prop-2-enyl 3-cyclohexylpropanoate; prop-2-enylheptanoate; (E)-1-(2,6,6-trimethyl-1-cyclohex-2-enyl)but-2-en-1-one; (E)-4-(2,6,6-trimethyl-1-cyclohex-2-enyl)but-3-en-2-one; (E)-3-methyl-4-(2,6,6-trimethyl-1-cyclohex-2-enyl)but-3-en-2-one; 1-(2,6,6-trimethyl-1-cyclohex-2-enyl)pent-1-en-3-one; 6,6,9a-trimethyl-1,2,3a,4,5,5a,7,8,9,9b-decahydronaphtho[2,1-b]furan; pentyl 2-hydroxybenzoate; 7,7-dimethyl-2-methylidene-norbornane; (E)-1-(2,6,6-trimethyl-1-cyclohexenyl)but-2-en-1-one; (E)-4-(2,6,6-trimethyl-1-cyclohexenyl)but-3-en-2-one; 4-ethoxy-4,8,8-trimethyl-9-methylidenebicyclo[3.3.1]nonane; (1,7,7-trimethyl-6-bicyclo[2.2.1]heptanyl) acetate; 3-(4-tert-butylphenyl)prop anal; 1,1,2,3,3-pentamethyl-2,5,6,7-tetrahydroinden-4-one; 2-oxabicyclo2.2.2octane,1methyl4(2,2,3trimethylcyclopentyl); [(Z)-hex-3-enyl]acetate; [(Z)-hex-3-enyl] 2-methylbutanoate; cis-3-hexenyl 2-hydroxybenzoate; 3,7-dimethylocta-2,6-dienal; 3,7-dimethyloct-6-en-1-al; 3,7-dimethyl-6-octen-1-ol; 3,7-dimethyloct-6-enyl acetate; 3,7-dimethyloct-6-enenitrile; 2-(3,7-dimethyloct-6-enoxy)acetaldehyde; tetrahydro-4-methyl-2-propyl-2h-pyran-4-yl acetate; ethyl 3-phenyloxirane-2-carboxylate; hexahydro-4,7-methano-indenyl isobutyrate; 2,4-dimethylcyclohex-3-ene-1-carbaldehyde; hexahydro-4,7-methano-indenyl propionate; 2-cyclohexylethyl acetate; 2-pentylcyclopentan-1-ol; (2R,3R,4S,5S,6R)-2-[(2R,3S,4R,5R,6R)-6-(6-cyclohexylhexoxy)-4,5-dihydroxy-2-(hydroxymethyl)oxan-3-yl]oxy-6-(hydroxymethyl)oxane-3,4,5-triol; (E)-1-(2,6,6-trimethyl-1-cyclohexa-1,3-dienyl)but-2-en-1-one; 1-cyclohexylethyl (E)-but-2-enoate; dodecanal; (E)-1-(2,6,6-trimethyl-1-cyclohex-3-enyl)but-2-en-1-one; (5E)-3-methylcyclopentadec-5-en-1-one; 4-(2,6,6-trimethyl-1-cyclohex-2-enyl)butan-2-one; 2-methoxy-4-propylphenol; methyl 2-hexyl-3-oxocyclopentane-1-carboxylate; 2,6-dimethyloct-7-en-2-ol; 4,7-dimethyloct-6-en-3-one; 4-(octahydro-4,7-methano-5H-inden-5-yliden)butanal; acetaldehyde ethyl linalyl acetal; ethyl 3,7-dimethyl-2,6-octadienoate; ethyl 2,6,6-trimethylcyclohexa-1,3-diene-1-carboxylate; 2-ethylhexanoate; (6E)-3,7-dimethylnona-1,6-dien-3-ol; ethyl 2-methylbutanoate; ethyl 2-methylpentanoate; ethyl tetradecanoate; ethyl nonanoate; ethyl 3-phenyloxirane-2-carboxylate; 1,4-dioxacycloheptadecane-5,17-dione; 1,3,3-trimethyl-2-oxabicyclo[2,2,2]octane; [essential oil]; oxacyclo-hexadecan-2-one; 3-(4-ethylphenyl)-2,2-dimethylpropanal; 2-butan-2-ylcyclohexan-1-one; 1,4-cyclohexandicarboxylic acid, diethyl ester; (3 aalpha,4beta,7beta,7aalpha)-octahydro-4,7-methano-3aH-indene-3a-carboxylic acid ethyl ester; hexahydro-4-7, menthano-1H-inden-6-yl propionate; 2-butenon-1-one,1-(2,6-dimethyl-6-methylencyclohexyl)-; (E)-4-(2,2-dimethyl-6-methylidenecyclohexyl)but-3-en-2-one; 1-methyl-4-propan-2-ylcyclohexa-1,4-diene; 5-heptyloxolan-2-one; 3,7-dimethylocta-2,6-dien-1-ol; [(2E)-3,7-dimethylocta-2,6-dienyl] acetate; [(2E)-3,7-dimethylocta-2,6-dienyl] octanoate; ethyl 2-ethyl-6,6-dimethylcyclohex-2-ene-1-carboxylate; (4-methyl-1-propan-2-yl-1-cyclohex-2-enyl) acetate; 2-butyl-4,6-dimethyl-5,6-dihydro-2H-pyran; oxacyclohexadecen-2-one; 1-propanol,2-[1-(3,3-dimethyl-cyclohexyl)ethoxy]-2-methylpropanoate; 1-heptyl acetate; 1-hexyl acetate; hexyl 2-methylpropanoate; (2-(1-ethoxyethoxy)ethyl)benzene; 4,4a,5,9b-tetrahydroindeno[1,2-d][1,3]dioxine; undec-10-enal; 3-methyl-4-(2,6,6-trimethyl-2-cyclohexen-1-yl)-3-buten-2-one; 1-(1,2,3,4,5,6,7,8-octahydro-2,3,8,8-tetramethyl-2-naphthalenyl)-ethan-1-one; 7-acetyl,1,2,3,4,5,6,7-octahydro-1,1,6,7,-tetra methyl naphthalene; 3-methylbutyl 2-hydroxybenzoate; [(1R,4S,6R)-1,7,7-trimethyl-6-bicyclo[2.2.1]heptanyl]acetate; [(1R,4R,6R)-1,7,7-trimethyl-6-bicyclo[2.2.1]heptanyl] 2-methylpropanoate; (1,7,7-trimethyl-5-bicyclo[2.2.1]heptanyl) propanoate; 2-methylpropyl hexanoate; [2-methoxy-4-[(E)-prop-1-enyl]phenyl] acetate; 2-hexylcyclopent-2-en-1-one; 5-methyl-2-propan-2-ylcyclohexan-1-one; 7-methyloctyl acetate; propan-2-yl 2-methylbutanoate; 3,4,5,6,6-pentamethylheptenone-2; hexahydro-3,6-dimethyl-2(3H)-benzofuranone; 2,4,4,7-tetramethyl-6,8-nonadiene-3-one oxime; dodecyl acetate; [essential oil]; 3,7-dimethylnona-2,6-dienenitrile; [(Z)-hex-3-enyl] methyl carbonate; 2-methyl-3-(4-tert-butylphenyl)propanal; 3,7-dimethylocta-1,6-dien-3-ol; 3,7-dimethylocta-1,6-dien-3-yl acetate; 3,7-dimethylocta-1,6-dien-3-yl butanoate; 3,7-dimethylocta-1,6-dien-3-yl formate; 3,7-dimethylocta-1,6-dien-3-yl 2-methylpropanoate; 3,7-dimethylocta-1,6-dien-3-yl propanoate; 3-methyl-7-propan-2-ylbicyclo[2.2.2]oct-2-ene-5-carbaldehyde; 2,2-dimethyl-3-(3-methylphenyl)propan-1-ol; 3-(4-tert-butylphenyl)butanal; 2,6-dimethylhept-5-enal; 5-methyl-2-propan-2-yl-cyclohexan-1-ol; 1-(2,6,6-trimethyl-1-cyclohexenyl)pent-1-en-3-one; methyl 3-oxo-2-pentylcyclopentaneacetate; methyl tetradecanoate; 2-methylundecanal; 2-methyldecanal; 1,1-dimethoxy-2,2,5-trimethyl-4-hexene; [(1S)-3-(4-methylpent-3-enyl)-1-cyclohex-3-enyl]methyl acetate; 2-(2-(4-methyl-3-cyclohexen-1-yl)propyl)cyclo-pentanone; 4-penten-1-one, 1-(5,5-dimethyl-1-cyclohexen-1-yl; 1H-indene-ar-propanal,2,3,-dihydro-1,1-dimethyl-(9CI); 2-ethoxynaphthalene; nonanal; 2-(7,7-dimethyl-4-bicyclo[3.1.1]hept-3-enyl)ethyl acetate; octanal; 4-(1-methoxy-1-methylethyl)-1-methylcyclohexene; (2-tert-butylcyclohexyl) acetate; (E)-1-ethoxy-4-(2-methylbutan-2-yl)cyclohexane; 1,1-dimethoxynon-2-yne; [essential oil]; 2-cyclohexylidene-2-phenylacetonitrile; 2-cyclohexyl-1,6-heptadien-3-one; 4-cyclohexyl-2-methylbutan-2-ol; 2-phenylethyl 2-phenylacetate; (2E, 5E/Z)-5,6,7-trimethyl octa-2,5-dien-4-one; 1-methyl-3-(4-methylpent-3-enyl)cyclohex-3-ene-1-carbaldehyde; methyl 2,2-dimethyl-6-methylidenecyclohexane-1-carboxylate; 1-(3,3-dimethylcyclohexyl)ethyl acetate; 4-methyl-2-(2-methylprop-1-enyl)oxane; 1-spiro(4.5)-7-decen-7-yl-4-penten-1-one; 4-(2-butenylidene)-3,5,5-trimethylcyclohex-2-en-1-one; 2-(4-methyl-1-cyclohex-3-enyl)propan-2-ol; 4-isopropylidene-1-methyl-cyclohexene; 2-(4-methyl-1-cyclohex-3-enyl)propan-2-yl acetate; 3,7-dimethyloctan-3-ol; 3,7-dimethyloctan-3-ol; 3,7-dimethyloctan-3-yl acetate; 3-phenylbutanal; (2,5-dimethyl-4-oxofuran-3-yl) acetate; 4-methyl-3-decen-5-ol; undec-10-enal; (4-formyl-2-methoxyphenyl) 2-methylpropanoate; 2,2,5-trimethyl-5-pentylcyclopentan-1-one; 2-tert-butylcyclohexan-1-ol; (2-tert-butylcyclohexyl) acetate; 4-tert-butylcyclohexyl acetate; 1-(3-methyl-7-propan-2-yl-6-bicyclo[2.2.2]oct-3-enyl)ethanone; (4,8-dimethyl-2-propan-2-ylidene-3,3a,4,5,6,8a-hexahydro-1H-azulen-6-yl) acetate; [(4Z)-1-cyclooct-4-enyl] methyl carbonate; methyl beta naphtyl ether; materials and stereoisomers thereof.

The compositions may also include a parent fragrance and one or more encapsulated fragrances that may or may not differ from the parent fragrance. For example, the composition may include a parent fragrance and a non-parent fragrance. A parent fragrance refers to a fragrance that is dispersed throughout the composition and is typically not encapsulated when added to the composition. Herein, a non-parent fragrance refers to a fragrance that differs from a parent fragrance included within the composition and is encapsulated with an encapsulating material prior to inclusion into the composition. Non-limiting examples of differences between a fragrance and a non-parent fragrance include differences in chemical make-up. In some examples, dried microcapsules may be incorporated into the hair conditioning composition, prepared by spray drying, fluid bed drying, tray drying, or other such drying processes that are available.

Conditioning Agent

The hair conditioning compositions disclosed herein may include a conditioning agent. The hair conditioning composition may include from 0.01% to 12% of a conditioning agent, by weight of the composition. Non-limiting examples of conditioning agents include cationic surfactants, high melting point fatty compounds, nonionic polymers, silicones, organic conditioning oils, and mixtures thereof.

### A. Cationic Surfactant

The conditioning agent for use in the compositions may contain a cationic surfactant. Any known cationic surfactant may be used herein. Examples include those surfactants disclosed in U.S. Patent (2009/0143267A1). Concentrations of cationic surfactant in the composition typically range from about 0.05% to about 3%, in other examples from about 0.075% to about 2.0%, alternatively from about 0.1% to about 1.0%.

A variety of cationic surfactants including mono- and di-alkyl chain cationic surfactants can be used in the conditioner composition. In some examples, mono-alkyl chain cationic surfactants are used in order to provide a consumer desired gel matrix and wet conditioning benefits. Such mono-alkyl cationic surfactants include, for example, mono-alkyl quaternary ammonium salts and mono-alkyl amines.

In some examples, cationic surfactants such as di-alkyl chain cationic surfactants are used in combination with mono-alkyl chain cationic surfactants. Such di-alkyl chain cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

Cationic surfactants can also be a salt of a mono-long alkyl quaternized ammonium and an anion, wherein the anion is selected from the group consisting of halides such as chloride and bromide, C1-C4 alkyl sulfate such as methosulfate and ethosulfate, and mixtures thereof. In some examples, the anion is selected from the group consisting of halides such as chloride.

The mono-long alkyl quaternized ammonium salts useful herein are those having the formula (I):

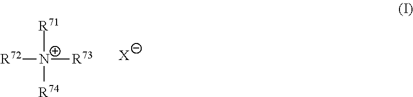

wherein one of R⁷¹, R⁷², R⁷³ and R⁷⁴ is selected from an aliphatic group of from 16 to 40 carbon atoms or an aromatic, alkoxy, polyoxyalkylene, alkylamido, hydroxyalkyl, aryl or alkylaryl group having up to about 40 carbon atoms; the remainder of R⁷¹, R⁷², R⁷³ and R⁷⁴ are independently selected from an aliphatic group of from 1 to about 8 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. In some examples, one of R⁷¹, R⁷², R⁷³ and R⁷⁴ is selected from an alkyl group of from 16 to 40 carbon atoms, alternatively from 18 to 26 carbon atoms, alternatively from 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Such mono-long alkyl quaternized ammonium salts provides an improved slippery feel to wet hair when compared to the slippery feeling produced by multi-long alkyl quaternized ammonium salts. In addition, mono-long alkyl quaternized ammonium salts provide improved hydrophobicity of the hair and give a smooth feel to dry hair, compared to amine or amine salt cationic surfactants.

In some examples, cationic surfactants are those having a longer alkyl group, i.e., C18-22 alkyl group. Such cationic surfactants include, for example, behenyl trimethyl ammonium chloride, methyl sulfate or ethyl sulfate, and stearyl trimethyl ammonium chloride, methyl sulfate or ethyl sulfate. In some examples, the cationic surfactants are behenyl trimethyl ammonium chloride, methyl sulfate or ethyl sulfate. In some examples, the cationic surfactants are behenyl trimethyl ammonium chloride. Cationic surfactants having a longer alkyl group provide improved deposition of microcapsules onto the hair thereby providing an increased amount of benefit agents on the hair. In addition, cationic surfactants having a longer alkyl group provide reduced irritation to the skin of the consumer compared to cationic surfactants having a shorter alkyl group.

Mono-alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; alternatively l-glutamic acid, lactic acid, citric acid. In some examples, the amines herein are partially neutralized with any of the acids at a molar ratio of the amine to the acid of from about 1:0.3 to about 1:2, or from about 1:0.4 to about 1:1.

### B. High Melting Point Fatty Compound

The conditioner agent for use in the conditioner composition may include a high melting point fatty compound. The high melting point fatty compound useful herein has a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section.

Among a variety of high melting point fatty compounds, fatty alcohols may be used. The fatty alcohols useful herein are those having from about 14 to about 30 carbon atoms, or even from about 16 to about 22 carbon atoms. These fatty alcohols are saturated and can be straight or branched chain alcohols. In one aspect, fatty alcohols include, for example, cetyl alcohol, stearyl alcohol, behenyl alcohol, and mixtures thereof.

In some examples, high melting point fatty compounds of a single compound of high purity are used. Single compounds of pure fatty alcohols are selected from the group consisting of pure cetyl alcohol, stearyl alcohol, and behenyl alcohol. By “pure” herein, what is meant is that the compound has a purity of at least about 90%, or even at least about 95%. These single compounds of high purity provide good rinsability from the hair when the consumer rinses off the composition.

The high melting point fatty compound is included in the composition at a level of from about 0.1% to about 40%, from about 1% to about 30%, from about 1.5% to about 16% by weight of the composition, or even from about 1.5% to about 8% in view of providing improved conditioning benefits such as slippery feel during the application to wet hair, softness and moisturized feel on dry hair.

### C. Nonionic Polymers

The conditioner agent for use in the conditioner composition may include a nonionic polymer. Polyalkylene glycols having a molecular weight of more than about 1000 are useful herein. Useful are those having the following general formula:

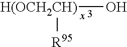

wherein R⁹⁵ is selected from the group consisting of H, methyl, and mixtures thereof. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

### D. Silicone Compound

The conditioner agent for use in the conditioner composition may include a silicone compound.

1. Silicones

The silicone compound may comprise volatile silicone, non-volatile silicones, or combinations thereof. In one aspect, non-volatile silicones are employed. If volatile silicones are present, it will typically be incidental to their use as a solvent or carrier for commercially available forms of non-volatile silicone materials ingredients, such as silicone gums and resins. The silicone compounds may comprise a silicone fluid conditioning agent and may also comprise other ingredients, such as a silicone resin to improve silicone fluid deposition efficiency or enhance glossiness of the hair.

The concentration of the silicone compound typically ranges from about 0.01% to about 10%, from about 0.1% to about 8%, from about 0.1% to about 5%, or even from about 0.2% to about 3%. Non-limiting examples of suitable silicone compounds, and optional suspending agents for the silicone, are described in U.S. Reissue Pat. No. 34,584, U.S. Pat. No. 5,104,646, and U.S. Pat. No. 5,106,609. The silicone compounds for use in the compositions typically have a viscosity, as measured at 25° C., from about 20 centistokes to about 2,000,000 centistokes (“cst”), from about 1,000 cst to about 1,800,000 cst, from about 50,000 cst to about 1,500,000 cst, or even from about 100,000 cst to about 1,500,000 cst.

The dispersed silicone compounds typically have a number average particle diameter ranging from about 0.01 μm to about 50 μm. For small particle application to hair, the number average particle diameters typically range from about 0.01 μm to about 4 μm, from about 0.01 μm to about 2 μm, or even from about 0.01 μm to about 0.5 μm. For larger particle application to hair, the number average particle diameters typically range from about 4 μm to about 50 μm, from about 6 μm to about 30 μm, from about 9 μm to about 20 μm, or even from about 12 μm to about 18 μm.

a. Silicone Oils

Silicone fluids may include silicone oils, which are flowable silicone materials having a viscosity, as measured at 25° C., less than 1,000,000 cst, from about 5 cst to about 1,000,000 cst, or even from about 100 cst to about 600,000 cst. Suitable silicone oils for use in the compositions include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

b. Amino and Cationic Silicones

Silicone compounds may include an aminosilicone. Aminosilicones, as provided herein, are silicones containing at least one primary amine, secondary amine, tertiary amine, or a quaternary ammonium group. Useful aminosilicones may have less than about 0.5% nitrogen by weight of the aminosilicone, less than about 0.2%, or even less than about 0.1%. Higher levels of nitrogen (amine functional groups) in the amino silicone tend to result in less friction reduction and consequently less conditioning benefit from the aminosilicone. It should be understood that in some product forms, higher levels of nitrogen are acceptable.

In some examples, the aminosilicones used may have a particle size of less than about 50μ once incorporated into the final composition. The particle size measurement is taken from dispersed droplets in the final composition. Particle size may be measured by means of a laser light scattering technique, using a Horiba model LA-930 Laser Scattering Particle Size Distribution Analyzer (Horiba Instruments, Inc.).

In some examples, the aminosilicone typically has a viscosity of from about 1,000 cst (centistokes) to about 1,000,000 cst, from about 10,000 to about 700,000 cst, from about 50,000 cst to about 500,000 cst, or even from about 100,000 cst to about 400,000 cst. These embodiments may also comprise a low viscosity fluid, such as, for example, those materials described below in Section F.(1). The viscosity of aminosilicones discussed herein is measured at 25° C.

In some examples, the aminosilicone typically has a viscosity of from about 1,000 cst to about 100,000 cst, from about 2,000 cst to about 50,000 cst, from about 4,000 cst to about 40,000 cst, or even from about 6,000 cst to about 30,000 cs.

In some examples, the aminosilicone is contained in the composition at a level by weight of from about 0.05% to about 20%, from about 0.1% to about 10%, and or even from about 0.3% to about 5%.

c. Silicone Gums

Other silicone compounds suitable for use in the compositions are the insoluble silicone gums. These gums are polyorganosiloxane materials having a viscosity, as measured at 25° C., of greater than or equal to 1,000,000 csk. Specific non-limiting examples of silicone gums for use in the compositions include polydimethylsiloxane, (polydimethylsiloxane) (methylvinylsiloxane) copolymer, poly(dimethylsiloxane) (diphenyl siloxane)(methylvinylsiloxane) copolymer and mixtures thereof.

d. High Refractive Index Silicones

Other non-volatile, insoluble silicone fluid compounds that are suitable for use in the compositions are those known as “high refractive index silicones,” having a refractive index of at least about 1.46, at least about 1.48, m at least about 1.52, or even at least about 1.55. The refractive index of the polysiloxane fluid will generally be less than about 1.70, typically less than about 1.60. In this context, polysiloxane “fluid” includes oils as well as gums.

The high refractive index polysiloxane fluid includes those represented by general Formula (III) above, as well as cyclic polysiloxanes such as those represented by Formula (VIII) below:

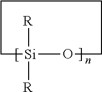

wherein R is as defined above, and n is a number from about 3 to about 7, or even from about 3 to about 5.

Silicone fluids suitable for use in the compositions are disclosed in U.S. Pat. No. 2,826,551, U.S. Pat. No. 3,964,500, and U.S. Pat. No. 4,364,837.

e. Silicone Resins

Silicone resins may be included in the conditioning agent of the compositions. These resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and must be specifically defined for each occurrence.

In some examples, silicone resins for use in the compositions include, but are not limited to MQ, MT, MTQ, MDT and MDTQ resins. In one aspect, Methyl is a highly suitable silicone substituent. In another aspect, silicone resins are typically MQ resins, wherein the M:Q ratio is typically from about 0.5:1.0 to about 1.5:1.0 and the average molecular weight of the silicone resin is typically from about 1000 to about 10,000.

f. Modified Silicones or Silicone Copolymers

Other modified silicones or silicone copolymers are also useful herein. Examples of these include silicone-based quaternary ammonium compounds (Kennan quats) disclosed in U.S. Pat. Nos. 6,607,717 and 6,482,969; end-terminal quaternary siloxanes; silicone aminopolyalkyleneoxide block copolymers disclosed in U.S. Pat. Nos. 5,807,956 and 5,981,681; hydrophilic silicone emulsions disclosed in U.S. Pat. No. 6,207,782; and polymers made up of one or more crosslinked rake or comb silicone copolymer segments disclosed in U.S. Pat. No. 7,465,439. Additional modified silicones or silicone copolymers useful herein are described in U.S. Patent Application Nos. 2007/0286837A1 and 2005/0048549A1.

In alternative embodiments, the above-noted silicone-based quaternary ammonium compounds may be combined with the silicone polymers described in U.S. Pat. Nos. 7,041,767 and 7,217,777 and U.S. Application number 2007/0041929A1.

The compositions herein may include a low viscosity silicone polymer having a viscosity up to 100,000 mPa·s. Structurally, the silicone polymer is a polyorganosiloxane compound comprising one or more quaternary ammonium groups, at least one silicone block comprising greater than 200 siloxane units, at least one polyalkylene oxide structural unit, and at least one terminal ester group. In one or more examples, the silicone block may comprise between 300 to 500 siloxane units.

The silicone polymer may be present in an amount of from about 0.05% to about 15%, alternatively from about 0.1% to about 10%, alternatively from about 0.15% to about 5%, and alternatively from about 0.2% to about 4% by weight of the composition.

In some examples, the polyorganosiloxane compounds have the general formulas (Ia) and (Ib):

M-Y—[—(N⁺R₂-T-N⁺R₂)—Y—]ₘ—[—(NR²-A-E-A′-NR²)—Y-]ₖ-M (Ia)

M-Y—[—(N⁺R₂-T-N⁺R₂)—Y—]ₘ—[—(N⁺R²₂-A-E-A′-N⁺R²₂)—Y-]ₖ-M (Ib)

wherein:

m is >0, preferred 0.01 to 100, more preferred 0.1 to 100, even more preferred 1 to 100, specifically 1 to 50, more specifically 1 to 20, even more specifically 1 to 10,

k is 0 or an average value of from >0 to 50, or preferably from 1 to 20, or even more preferably from 1 to 10,

M represents a terminal group, comprising terminal ester groups selected from

### —OC(O)—Z

### —OS(O)₂—Z

### —OS(O₂)O—Z

### —OP(O)(O—Z)OH

### —OP(O)(O—Z)₂

wherein Z is selected from monovalent organic residues having up to 40 carbon atoms, optionally comprising one or more hetero atoms.

A and A′ each are independently from each other selected from a single bond or a divalent organic group having up to 10 carbon atoms and one or more hetero atoms, and

E is a polyalkylene oxide group of the general formula:

—[CH₂CH₂O]_{q}—[CH₂CH(CH₃)O]ᵣ—[CH₂CH(C₂H₅)O]ₛ—

wherein q=0 to 200, r=0 to 200, s=0 to 200, and q+r+s=1 to 600.

R² is selected from hydrogen or R,

R is selected from monovalent organic groups having up to 22 carbon atoms and optionally one or more heteroatoms, and wherein the free valencies at the nitrogen atoms are bound to carbon atoms,

Y is a group of the formula:

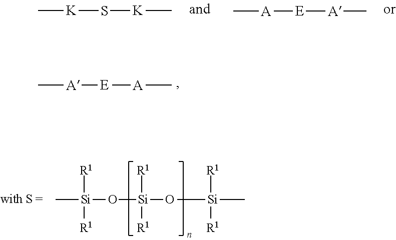

wherein R1=C₁-C₂₂-alkyl, C₁-C₂₂-fluoroalkyl or aryl; n=200 to 1000, and these can be identical or different if several S Groups are present in the polyorganosiloxane compound.

K is a bivalent or trivalent straight chain, cyclic and/or branched C₂-C₄₀ hydrocarbon residue which is optionally interrupted by —O—, —NH—, trivalent N, —NR¹—, —C(O)—, —C(S)—, and optionally substituted with —OH, wherein R¹ is defined as above,

T is selected from a divalent organic group having up to 20 carbon atoms and one or more hetero atoms.

The residues K may be identical or different from each other. In the —K—S—K— moiety, the residue K is bound to the silicon atom of the residue S via a C—Si-bond.

Due to the possible presence of amine groups (—(NR²-A-E-A′-NR²)—) in the polyorganosiloxane compounds, they may have protonated ammonium groups, resulting from the protonation of such amine groups with organic or inorganic acids. Such compounds are sometimes referred to as acid addition salts of the polyorganosiloxane compounds herein.

In some examples, the molar ratio of the quaternary ammonium groups b) and the terminal ester groups c) is less than 100:20, alternatively less than 100:30, and alternatively less than 100:50. The ratio can be determined by ¹³C-NMR.

In some examples, the polyorganosiloxane composition may comprise:

A) at least one polyorganosiloxane compound, comprising a) at least one polyorganosiloxane group, b) at least one quaternary ammonium group, c) at least one terminal ester group, and d) at least one polyalkylene oxide group (as defined before),

B) at least one polyorganosiloxane compound, comprising at least one terminal ester group, different from compound A).

In the definition of component A) it can be referred to the description of the polyorganosiloxane compounds. The polyorganosiloxane compound B) differs from the polyorganosiloxane compound A) preferably in that it does not comprise quaternary ammonium groups. Preferred polyorganosiloxane compounds B) result from the reaction of monofunctional organic acids, in particular carboxylic acids, and polyorganosiloxane containing bisepoxides.

In the polyorganosiloxane compositions herein the weight ratio of compound A) to compound B) is preferably less than 90:10. Or in other words, the content of component B) is at least 10 weight percent. In some examples, the polyorganosiloxane compositions in compound A) the molar ratio of the quaternary ammonium groups b) and the terminal ester groups c) is less than 100:10, even more preferred is less than 100:15 and is most preferred less than 100:20.

The silicone polymer has a viscosity at 20° C. and a shear rate of 0.1s⁻¹ (plate-plate system, plate diameter 40 mm, gap width 0.5 mm) of less than 100,000 mPa·s (100 Pa·s). In some examples, the viscosities of the neat silicone polymers may range from 500 to 100,000 mPa·s, or preferably from 500 to 70,000 mPa·s, or more preferably from 500 to 50,000 mPa·s, or even more preferably from 500 to 20,000 mPa·s. In some examples, the viscosities of the neat polymers may range from 500 to 10,000 mPa·s, or preferably 500 to 5000 mPa·s determined at 20° C. and a shear rate of 0.1 s⁻¹.

In addition to the above listed silicone polymers, the following preferred compositions are provided below. For example, in the polyalkylene oxide group E of the general formula:

—[CH₂CH₂O]_{q}—[CH₂CH(CH₃)O]ᵣ—[CH₂CH(C₂H₅)O]ₛ—

wherein the q, r, and s indices may be defined as follows:

q=0 to 200, or preferably from 0 to 100, or more preferably from 0 to 50, or even more preferably from 0 to 20,

r=0 to 200, or preferably from 0 to 100, or more preferably from 0 to 50, or even more preferably from 0 to 20,

s=0 to 200, or preferably from 0 to 100, or more preferably from 0 to 50, or even more preferably from 0 to 20,

and q+r+s=1 to 600, or preferably from 1 to 100, or more preferably from 1 to 50, or even more preferably from 1 to 40.

For polyorganosiloxane structural units with the general formula S:

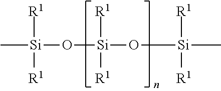

R¹=C₁-C₂₂-alkyl, C₁-C₂₂-fluoralkyl or aryl; n=from 200 to 1000, or preferably from 300 to 500, K (in the group —K—S—K—) is preferably a bivalent or trivalent straight chain, cyclical or branched C₂-C₂₀ hydrocarbon residue which is optionally interrupted by —O—, —NH—, trivalent N, —NR¹—, —C(O)—, —C(S)—, and optionally substituted with —OH.

In some examples, R¹ is C₁-C₁₈ alkyl, C₁-C₁₈ fluoroalkyl and aryl. Furthermore, R¹ is preferably C₁-C₁₈ alkyl, C₁-C₆ fluoroalkyl and aryl. Furthermore, R¹ is more preferably C₁-C₆ alkyl, C₁-C₆ fluoroalkyl, even more preferably C₁-C₄ fluoroalkyl, and phenyl. Most preferably, R¹ is methyl, ethyl, trifluoropropyl and phenyl.

As used herein, the term “C₁-C₂₂ alkyl” means that the aliphatic hydrocarbon groups possess from 1 to 22 carbon atoms which can be straight chain or branched. Methyl, ethyl, propyl, n-butyl, pentyl, hexyl, heptyl, nonyl, decyl, undecyl, isopropyl, neopentyl and 1,2,3-trimethyl hexyl moieties serve as examples.

Further as used herein, the term “C₁-C₂₂ fluoroalkyl” means aliphatic hydrocarbon compounds with 1 to 22 carbon atoms which can be straight chain or branched and are substituted with at least one fluorine atom. Monofluormethyl, monofluoroethyl, 1,1,1-trifluorethyl, perfluoroethyl, 1,1,1-trifluoropropyl, 1,2,2-trifluorobutyl are suitable examples.

Moreover, the term “aryl” means unsubstituted or phenyl substituted once or several times with OH, F, Cl, CF₃, C₁-C₆ alkyl, C₁-C₆alkoxy, C₃-C₇ cycloalkyl, C₂-C₆ alkenyl or phenyl. Aryl may also mean naphthyl.

In some examples using the polyorganosiloxanes, the positive charges resulting from the ammonium group(s), are neutralized with inorganic anions such as chloride, bromide, hydrogen sulfate, sulfate, or organic anions, like carboxylates deriving from C₁-C₃₀ carboxylic acids, for example acetate, propionate, octanoate, especially from C₁₀-C₁₈ carboxylic acids, for example decanoate, dodecanoate, tetradecanoate, hexadecanoate, octadecanoate and oleate, alkylpolyethercarboxylate, alkylsulphonate, arylsulphonate, alkylarylsulphonate, alkylsulphate, alkylpolyethersulphate, phosphates derived from phosphoric acid mono alkyl/aryl ester and phosphoric acid dialkyl/aryl ester. The properties of the polyorganosiloxane compounds can be, inter alia, modified based upon the selection of acids used.

The quaternary ammonium groups are usually generated by reacting the di-tertiary amines with an alkylating agents, selected from in particular di-epoxides (sometimes referred to also as bisepoxides) in the presence of mono carboxylic acids and difunctional dihalogen alkyl compounds.

In some examples, the polyorganosiloxane compounds are of the general formulas (Ia) and (Ib):

M-Y—[—(N⁺R₂-T-N⁺R₂)—Y—]ₘ—[—(NR²-A-E-A′-NR²)—Y-]ₖ-M (Ia)

M-Y—[—(N⁺R₂-T-N⁺R₂)—Y—]ₘ—[—(N⁺R²₂A-E-A′-N⁺R²₂)—Y-]ₖ-M (Ib)

wherein each group is as defined above; however, the repeating units are in a statistical arrangement (i.e., not a block-wise arrangement).

In some examples, the polyorganosiloxane compounds may be also of the general formulas (IIa) or (IIb):

M-Y—[—N⁺R₂—Y—]ₘ—[—(NR²-A-E-A′-NR²)—Y-]ₖ-M (IIa)

M-Y—[—N⁺R₂—Y—]ₘ—[—(N⁺R²₂-A-E-A′-N⁺R²₂)—Y-]ₖ-M (IIb)

wherein each group is as defined above. Also in such formula the repeating units are usually in a statistical arrangement (i.e not a block-wise arrangement).

wherein, as defined above, M is

### —OC(O)—Z,

### —OS(O)₂—Z

### —OS(O₂)O—Z

### —OP(O)(O—Z)OH

### —OP(O)(O—Z)₂

Z is a straight chain, cyclic or branched saturated or unsaturated C₁-C₂₀, or preferably C₂ to C₁₈, or even more preferably a hydrocarbon radical, which can be interrupted by one or more —O—, or —C(O)— and substituted with —OH. In some examples, M is —OC(O)—Z resulting from normal carboxylic acids in particular with more than 10 carbon atoms like for example dodecanoic acid.

In some examples, the molar ratio of the polyorganosiloxane-containing repeating group K—S—K— and the polyalkylene repeating group -A-E-A′- or -A′-E-A- is between 100:1 and 1:100, or preferably between 20:1 and 1:20, or more preferably between 10:1 and 1:10.

In the group —(N⁺R₂-T-N⁺R₂)—, R may represent a monovalent straight chain, cyclic or branched C₁-C₂₀ hydrocarbon radical, which can be interrupted by one or more —O—, —C(O)— and can be substituted by —OH, T may represent a divalent straight-chain, cyclic, or branched C₁-C₂₀ hydrocarbon radical, which can be interrupted by —O—, —C(O)— and can be substituted by hydroxyl.

The above described polyorganosiloxane compounds comprising quaternary ammonium functions and ester functions may also contain: 1) individual molecules which contain quaternary ammonium functions and no ester functions; 2) molecules which contain quaternary ammonium functions and ester functions; and 3) molecules which contain ester functions and no quaternary ammonium functions. While not limited to structure, the above described polyorganosiloxane compounds comprising quaternary ammonium functions and ester functions are to be understood as mixtures of molecules comprising a certain averaged amount and ratio of both moieties.

Various monofunctional organic acids may be utilized to yield the esters. Exemplary embodiments include C₁-C₃₀ carboxylic acids, for example C₂, C₃, C₈ acids, C₁₀-C₁₈ carboxylic acids, for example C₁₂, C₁₄, C₁₆ acids, saturated, unsaturated and hydroxyl functionalized C₁₈ acids, alkylpolyethercarboxylic acids, alkylsulphonic acids, arylsulphonic acids, alkylarylsulphonic acids, alkylsulphuric acids, alkylpolyethersulphuric acids, phosphoric acid mono alkyl/aryl esters and phosphoric acid dialkyl/aryl esters.

Further performance improvements may optionally be achieved by pre-dispersing the silicone polymer in a small particle emulsion (less than 1 micron) prior to adding it to the conditioner base.

The term “emulsion” in this patent application describes any stable emulsion or dispersion of the silicone polymer, separately prepared and used as one of the components of a conditioner composition.

Stable means that the viscosity, particle size, and other important characteristics of the emulsion do not significantly change over reasonable time under exposure to typical temperature, moisture, pressure, shear, light and other environmental conditions that the pre-emulsion is exposed during packing, storage, and transportation

Making the small particle emulsion may require pre-emulsifying the silicone polymer before their addition to the conditioning composition. A non-limiting example of a method of making is provided below. All oil soluble components are mixed in a vessel. Heat may be applied to allow mixture to liquidify. All water soluble components are mixed in a separate vessel and heated to about same temperature as the oil phase. The oil phase and aqueous phase are mixed under a high shear mixer (example, Turrax mixer by IKA). The particle size of the silicone polymer is in the range of about 0.01 μm to about 5 μm, alternatively from 0.05 μm to about 1 μm, alternatively from about 0.1 μm to about 0.5 μm. High energy mixing device may be used to achieve desired particle size. High energy mixing device include, but not limited to Microfluidizer from Microfluidics Corp., Sonolator from Sonic Corp., Colloid mill from Sonic Corp.

The emulsifiers which may be selected for each the silicone may be guided by the Hydrophilic-Lipophilic-Balance value (HLB value) of emulsifiers. Suitable range of HLB value may be 6-16, alternatively 8-14. Emulsifiers with a HLB higher than 10 are water soluble. Emulsifiers with low HLB are lipid soluble. To obtain suitable HLB value, a mixture of two or more emulsifiers may be used. Suitable emulsifiers include non-ionic, cationic, anionic and amphoteric emulsifiers.

The concentration of the emulsifier in the emulsion and the emulsifications of the silicone polymer should be sufficient to achieve desired particle size and emulsion stability, and generally may range from about 0.1 wt %-about 50 wt %, from about 1 wt %-about 30 wt %, from about 2 wt %-about 20 wt %, for example.

The optional use of a pre-emulsified dispersion of the silicone may present multiple advantages including: (i) The small particle size of the silicones in the emulsion leads to more even deposition and reduces island-like spotty deposits; and (ii) the more even deposition is more favorable for providing smoothness for hair/skin surfaces, easier combing, and enhanced hair volume.

2. Organic Conditioning Oils

The compositions may also comprise from about 0.05% to about 3%, from about 0.08% to about 1.5%, or even from about 0.1% to about 1%, of at least one organic conditioning oil as the conditioning agent, either alone or in combination with other conditioning agents, such as the silicones (described herein). Suitable conditioning oils include hydrocarbon oils, polyolefins, and fatty esters. Suitable hydrocarbon oils include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils are typically from about C₁₂ to about C₁₉. Branched chain hydrocarbon oils, including hydrocarbon polymers, typically will contain more than 19 carbon atoms. Suitable polyolefins include liquid polyolefins, liquid poly-α-olefins, or even hydrogenated liquid poly-α-olefins. Polyolefins for use herein may be prepared by polymerization of C₄ to about C₁₄ or even C₆ to about C₁₂. Suitable fatty esters include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

3. Other Conditioning Agents

Hair conditioning agents may also comprise hydrolysed collagen with tradename Peptein 2000 available from Hormel, water soluble and water insoluble vitamins such as vitamin A, D, B₁, B₂, B₆, B₁₂, C, biotin, vitamin E with tradename Emix-d available from Eisai, panthenol available from Roche, pantothenic acid, panthenyl ethyl ether available from Roche, and their derivatives; hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose, Urea, Niacinamide, Glucosamine, Inositol, Sodium Benzoate, and Lactic acid) available from Goldschmidt, Sodium lactate, Sodium PCA, Glycine, Fructose, Urea, Niacinamide, Glucosamine, Inositol, Sodium Benzoate, Lactic acid, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, silicone grafted copolymers, and combinations thereof. Also suitable for use in the compositions herein are the conditioning agents described by the Procter & Gamble Company in U.S. Pat. Nos. 5,674,478, and 5,750,122. Also suitable for use herein are those conditioning agents described in U.S. Pat. Nos. 4,529,586, 4,507,280, 4,663,158, 4,197,865, 4,217, 914, 4,381,919, and 4,422, 853.

### E. Rheology Modifier

In some examples, the hair conditioning compositions may include rheology modifiers to adjust the rheological characteristics of the composition for better feel, in-use properties and the suspending stability of the composition. For example, the rheological properties are adjusted so that the composition remains uniform during its storage and transportation and it does not drip undesirably onto other areas of the body, clothing or home furnishings during its use. Any suitable rheology modifier can be used. In some examples, the hair conditioning composition may comprise from about 0.01% to about 3% of a rheology modifier, alternatively from about 0.1% to about 1% of a rheology modifier,

The rheology modifier may be a polyacrylamide thickener, a cationically modified polysaccharide, or an associative thickeners. Associative thickeners is an important class of rheology modifiers. It includes a variety of material classes such as, for example: hydrophobically modified cellulose derivatives; hydrophobically modified alkoxylated urethane polymers, such as PEG-150/decyl alcohol/SMDI copolymer, PEG-150/stearyl alcohol/SMDI copolymer, polyurethane-39; hydrophobically modified, alkali swellable emulsions, such as hydrophobically modified polypolyacrylates, hydrophobically modified polyacrylic acids, and hydrophobically modified polyacrylamides; hydrophobically modified polyethers. The class of materials includes numerous members. Typically these materials have a hydrophobe that can be selected from cetyl, stearyl, oleayl, and combinations thereof, and a hydrophilic portion of repeating ethylene oxide groups with repeat units from 10-300, more preferably from 30-200, more preferably from 40-150. Examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

Rheology modifiers useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carrageenan, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Other optional suspending agents include crystalline suspending agents which can be categorized as acyl derivatives, long chain amine oxides, and mixtures thereof. These suspending agents are described in U.S. Pat. No. 4,741,855.

These rheology modifiers include ethylene glycol esters of fatty acids in one aspect having from about 16 to about 22 carbon atoms. In one aspect, useful rheology modifiers include ethylene glycol stearates, both mono and distearate, but in one aspect, the distearate containing less than about 7% of the mono stearate. Other suitable rheology modifiers include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® R available from Rheox, Inc. Long chain acyl derivatives, ethylene glycol esters of long chain carboxylic acids, long chain amine oxides, and alkanol amides of long chain carboxylic acids in addition to the materials listed above may be used as rheology modifiers.

Other long chain acyl derivatives suitable for use as rheology modifiers include N,N-dihydrocarbyl amido benzoic acid and soluble salts thereof (e.g., Na, K), particularly N,N-di(hydrogenated) C₁₆, C₁₈ and tallow amido benzoic acid species of this family, which are commercially available from Stepan Company (Northfield, Ill., USA).

Examples of suitable long chain amine oxides for use as rheology modifiers include alkyl dimethyl amine oxides, e.g., stearyl dimethyl amine oxide.

Other suitable rheology modifiers include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable rheology modifiers include di(hydrogenated tallow)phthalic acid amide, and crosslinked maleic anhydride-methyl vinyl ether copolymer.

Non-limiting examples of rheology modifiers include acrylamide/ammonium acrylate copolymer (and) polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-2 hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquaternium-37 (and) hydrogenated polydecene (and) trideceth-6; polyurethane-39; sodium acrylate/acryloyldimethyltaurate/dimethylacrylamide; crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™ 33, ACULYN™ 46, ACULYN™ 22, ACULYN™ 44, Carbopol Ultrez 20, Carbopol Ultrez 21, Carbopol Ultrez 10, Carbopol 1342, Sepigel™ 305, Simulgel™ 600, Sepimax Zen, and/or combinations thereof. Commercially available viscosity modifiers highly useful herein include Carbomers with trade names Carbopol® 934, Carbopol® 940, Carbopol® 950, Carbopol® 980, and Carbopol® 981, all available from B. F. Goodrich Company, acrylates/steareth-20 methacrylate copolymer with trade name ACRYSOL™ 22 available from Rohm and Hass, nonoxynyl hydroxyethylcellulose with trade name Amercell™ POLYMER HM-1500 available from Amerchol, methylcellulose with trade name BENECEL®, hydroxyethyl cellulose with trade name NATROSOL®, hydroxypropyl cellulose with trade name KLUCEL®, cetyl hydroxyethyl cellulose with trade name POLYSURF® 67, all supplied by Hercules, ethylene oxide and/or propylene oxide based polymers with trade names CARBOWAX® PEGs, POLYOX WASRs, and UCON® FLUIDS, all supplied by Amerchol.

### F. Gel Matrix

The above cationic surfactants, together with high melting point fatty compounds and an aqueous carrier, may form a gel matrix in the composition.

The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, from about 1:1 to about 1:10, or even from about 1:1 to about 1:6.

### Carrier

Hair conditioning compositions typically comprise a carrier, which may be present at a level of from about 20 wt % to about 99 wt %, and/or from about 60 wt % to about 85 wt %. The carrier may comprise water, organic solvents (miscible or non-miscible with water), silicone solvents and/or mixtures thereof. The solvents should be dermatologically acceptable. The carrier may not comprise more than about 2, about 1, about 0.5, about 0.2, about 0.1, and/or about 0.05 wt % of non-volatile solvent. Significantly higher concentration of non-volatile carrier will increase hair weigh-down. and greasy hair feel. In some examples, the carrier may comprise water with minimal or no significant concentrations of organic solvent, except as otherwise incidentally incorporated into the composition as minor ingredients of other components. Water, organic and silicone solvents that have boiling points below or equal to 250° C. are volatile solvents. Solvents with boiling points above 250° C. are considered non-volatile.

The non-limiting examples of carriers include water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include glycols, glycerine and other diols.

Other Components

The composition may include other components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes, oxidative dyes and interference pigments; hair oxidizing (bleaching) agents, such as hydrogen peroxide, perborate and persulfate salts, carbonate; hair reducing agents such as the thioglycolates; perfumes; and sequestering agents, such as disodium ethylenediamine tetra-acetate; ultraviolet and infrared screening and absorbing agents such as octyl salicylate; antimicrobial agents; suspending agents; viscosity modifiers; nonvolatile solvents or diluents (water soluble and insoluble), pearlescent aids, foam boosters, additional surfactants or nonionic cosurfactants, pediculocides, chelants, skin active agents, sunscreens, UV absorbers, and, water soluble and insoluble amino acids such as asparagine, alanin, indole, glutamic acid, tyrosine, tryptamine, and their salts; and antidandruff agents such as zinc pyrithione, pyridinethione salts, azoles, climbazole, octopirox, salicylic acid, selenium sulfide, particulate sulfur, mixtures thereof.

Polysorbate

The hair conditioning compositions herein may contain a polysorbate, in view of adjusting rheology. Preferred polysorbate useful herein includes, for example, polysorbate-20, polysorbate-21, polysorbate-40, polysorbate-60, and mixtures thereof. Highly preferred is polysorbate-20.

The polysorbate can be contained in the composition at a level by weight of preferably from about 0.01% to about 5%, more preferably from about 0.05% to about 2%.

Polypropylene Glycol

Polypropylene glycol useful herein are those having a weight average molecular weight of from about 200 g/mol to about 100,000 g/mol, preferably from about 1,000 g/mol to about 60,000 g/mol. Without intending to be limited by theory, it is believed that the polypropylene glycol herein deposits onto, or is absorbed into hair to act as a moisturizer buffer, and/or provides one or more other desirable hair conditioning benefits.

The polypropylene glycol useful herein may be either water-soluble, water-insoluble, or may have a limited solubility in water, depending upon the degree of polymerization and whether other moieties are attached thereto. The desired solubility of the polypropylene glycol in water will depend in large part upon the form (e.g., leave-on, or rinse-off form) of the hair conditioning composition. For example, a rinse-off hair conditioning composition, it is preferred that the polypropylene glycol herein has a solubility in water at about 25° C. of less than about 1 g/100 g water, more preferably a solubility in water of less than about 0.5 g/100 g water, and even more preferably a solubility in water of less than about 0.1 g/100 g water.

The polypropylene glycol can be included in the hair conditioning composition at a level of, preferably from about 0.01% to about 10%, more preferably from about 0.05% to about 6%, still more preferably from about 0.1% to about 3% by weight of the composition.

Low Melting Point Oil

Low melting point oils useful herein are those having a melting point of less than about 25° C. The low melting point oil useful herein is selected from the group consisting of: hydrocarbon having from about 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof,

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Particularly useful citrate ester oils herein include triisocetyl citrate with tradename CITMOL 316 available from Bernel, triisostearyl citrate with tradename PELEMOL TISC available from Phoenix, and trioctyldodecyl citrate with tradename CITMOL 320 available from Bernel.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

Cationic Polymer

Cationic polymers useful herein are those having a weight average molecular weight of at least about 5,000, typically from about 10,000 to about 10 million, preferably from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

Polyethylene Glycol

Polyethylene glycol can also be used as an additional component. The polyethylene glycols useful herein that are especially preferred are PEG-2M wherein n has an average value of about 2,000 (PEG-2M is also known as Polyox WSR® N-10 from Union Carbide and as PEG-2,000); PEG-5M wherein n has an average value of about 5,000 (PEG-5M is also known as Polyox WSR® N-35 and as Polyox WSR® N-80, both from Union Carbide and as PEG-5,000 and Polyethylene Glycol 300,000); PEG-7M wherein n has an average value of about 7,000 (PEG-7M is also known as Polyox WSR® N-750 from Union Carbide); PEG-9M wherein n has an average value of about 9,000 (PEG-9M is also known as Polyox WSR® N-3333 from Union Carbide); and PEG-14M wherein n has an average value of about 14,000 (PEG-14M is also known as Polyox WSR® N-3000 from Union Carbide). As used herein “n” refers to the number of ethylene oxide units in the polymer.

### Method of Making Conditioner Formulations

The hair conditioning compositions may be prepared by any conventional method well known in the art. They are suitably made as follows: deionized water is heated to 85° C. and cationic surfactants and high melting point fatty compounds are mixed in. If necessary, cationic surfactants and fatty alcohols can be pre-melted at 85° C. before addition to the water. The water is maintained at a temperature of about 85° C. until the components are homogenized, and no solids are observed. The mixture is then cooled to about 55° C. and maintained at this temperature, to form a gel matrix. Silicones, or a blend of silicones and a low viscosity fluid, or an aqueous dispersion of a silicone is added to the gel matrix. When included, poly alpha-olefin oils, polypropylene glycols, and/or polysorbates are also added to the gel matrix. When included, other additional components such as perfumes and preservatives are added with agitation. The gel matrix is maintained at about 50° C. during this time with constant stirring to assure homogenization. After it is homogenized, it is cooled to room temperature. A triblender and/or mill can be used in each step, if necessary to disperse the materials.

### Method of Use

The hair conditioning compositions disclosed herein may be used in conventional ways to provide conditioning and other benefits. Such method of use depends upon the type of composition employed, but generally involves application of an effective amount as disclosed herein of the product to the hair or scalp, which may then be rinsed from the hair or scalp (as in the case of hair rinses) or allowed to remain on the hair or scalp (e.g. a leave-on conditioner in the form of a gel, lotion, cream, or spray).

### Product Forms

The hair conditioning compositions disclosed herein may be in the form of rinse-off products or leave-on products, can be opaque, and can be formulated in a wide variety of product forms, including but not limited to creams, gels, emulsions, mousses and sprays. However, the hair conditioning composition disclosed herein is preferably a leave-on product.

The hair conditioning composition may optionally relate to aqueous emulsions comprising at least one polyorganosiloxane compound and/or at least one polyorganosiloxane composition as defined above. Such aqueous emulsions preferably comprise at least 30 weight percent, preferably at least 50 weight percent, still more preferably at least 80 weight percent water based on the total weight of the emulsions.

The hair conditioning compositions disclosed herein may be suitable for rinse-off products and leave-on products.

### Treatment

One of skill in the art will understand that controlling the dosage applied by a user of a hair conditioning composition will vary depending on the type of container (pump dispenser vs. jar) and the form of the product (e.g. spray vs. gel) involved. Often, the container will be sealed. In some examples, the container will be a dispenser configured to release a consistent amount of the product per actuation. In some examples, when a dispenser that is configured to release a consistent amount of the product per actuation is used to store the hair conditioning composition, an instruction to the user may accompany the dispenser as to instruct the user as to how many actuations are required per application in order to arrive at the desired dose. A non-limiting example of such an instruction is “Apply 1-2 pumps to hair—starting at the bottom and working your way up—stopping about mid shaft. No need to wait or even rinse—go right to bed!” Another non-limiting example of such an instruction is “Place one to two spritzes into the palm of the hands, run gently through the dry hair. For wet hair, apply three to four spritzes.”

When hair conditioning composition is sold in a dispenser that is not configured to release a consistent amount of the product per actuation, non-limiting examples of which include jars and tottles, an instruction may accompany the product sold so as to instruct the user of the appropriate amount to apply. In some examples, the instruction may instruct a user on the dosage by instructing to apply an amount equal or similar to that of the size of a common household item, non-limiting examples of which include walnuts and quarters. A non-limiting example of such an instruction is “Starting with the ends and spreading throughout the lengths of the hair, apply a walnut-sized amount to dry hair. Massage the lengths and ends, applying more to the driest areas. Comb, but do not rinse.” Another non-limiting example of such an instruction includes “Apply a quarter-sized amount of the composition to palms and rub into hair. Do not rinse.”

### Test Methods

### Hair Switch Treatment Method

a) Hair Type: General population hair, moderately damaged, International Hair Importers.
b) Hair Switch Sizes: 4 gram/8 inch or 10 gram/10 inch
Store hair in foil, tissue paper, or Kimwipes, not paper towels. Paper towels can contain silicone, therefore, are not used to avoid any additional contamination of the hair.

Test Set-Up: Preparation/Labeling

Hair Switches
Depending on the number of switches per product that is requested (standard is 2), hair switches should be labeled to correspond with the product sample codes. Marked switches are then hung on a cart in corresponding order.

Switch Treatment Conditions:
1. Constant Water Temperature
A temperature gauge should be installed at the sink to ensure a constant temperature throughout the treatment portion of the test. The standard temperature should be set at 100 degrees F.+/−3 degrees F.
2. Constant Water Pressure
The pressure of the rinse water must be regulated for rinsing consistency. A flow meter should be installed at the sink and adjusted to standard measurement of 1.5 gallons per minute+/−0.25 gpm.
3. Water Hardness—an average of 7-13 grain.
4. Milking and Rinsing Guidelines—milk at a rate of 50-60 strokes per 30 seconds. The milking procedure (stroking motion from top to bottom) is very critical to achieving consistent results from each switch within the confines of a product. A consistent milking pattern, maintaining that pattern and a constant rhythm throughout the treatment of all switches is critical. Milk the switch hard enough to allow the product to come in contact with the hair through its thickness and not just the outer layers.
5. A stationary shower rinse is used with no additional manipulation of the hair for 30 seconds. Lightly squeegee twice down the switch from top to bottom between fingers after rinsing to remove excess water.

Treatment Procedure—1 Cycle
1) Requirement: wear vinyl gloves during the treatment process changing between every switch
2) Use a separate 1 cc disposable syringe for each product application
3) Standard product amount: 0.1 cc (equivalent to approximately 0.1 grams) per gram of hair
4) Avoid contamination: handle switches by taped top and avoid contact with other switches/surfaces
5) Pull up required product amount into syringes for each test product (make sure no air bubbles are in the syringe)
6) Apply conditioner product (0.10 cc per gram of hair) evenly from top to bottom starting 1 inch down from the clamp using a 1 ml disposable syringe
   a. Milk 50-60 strokes/30 seconds.
   b. Then hang on drying cart at ambient temperature and approximately 30% relative humidity.

### Olfactive Analysis Method

Once switches have been treated according to the Hair Switch Treatment method, and allowed to dry for at least 4 hours at 70° F./30% RH:
1) A perfumer or trained panelist assesses the fragrance on the hair switch by bringing the middle portion of the hair switch to the nose, and making an olfactive assessment. The Primavera olfactive grade is recorded as “initial pre-comb”.
2) Next, a perfumer or trained panelist combs the hair switch 3× with the fine tooth side of a comb on both sides of the hair (11 cm long—teeth to teeth, 1.5 cm long teeth, teeth spaced approximately 0.10 cm apart), and then brings the middle portion of the hair switch to the nose, and makes an olfactive assessment. The Primavera olfactive grade is recorded as “initial post-comb”.
3) In this manner, multiple combing sequences can be completed at different time points, using the same hair switch, in order to collect perfume intensity data.
4) The olfactive intensity scale ratings are given below.

| | Concentration of | |
|---|---|---|
| | DihydroMyrcenol in | |
| Olfactive Grade | mineral oil | Descriptors |
| 0 | 0% | No Odor |
| 25 | 0.005% | Weak |
| 50 | 0.2% | Moderate |
| 75 | 2% | Strong |
| 100 | 100% | Very Strong |

A difference of 5 points on this scale is not considered a noticeable difference on hair. A 10 point difference in olfactive grade is large and noticeable.

### NON-LIMITING EXAMPLES

The compositions illustrated in the following examples and tables exemplify specific embodiments of the compositions of the provided disclosure, but are not intended to be limiting thereof. Other modifications may be undertaken by the skilled artisan without departing from the spirit and scope of this invention.

The compositions illustrated in the following examples are prepared by conventional formulation and mixing methods, an example of which is described below. All exemplified amounts are listed as weight percents and exclude minor materials such as diluents, preservatives, color solutions, imagery ingredients, botanicals, and so forth, unless otherwise specified.

Perfume Oils utilized in encapsulation preferably have a octanol-water partition coefficient C log P greater than 1.5, and a boiling point greater than 130 degrees Celsius.

### Example 1

### Perfume Microcapsules

An oil solution, consisting of 128.4 g of perfume oil A, 32.1 g isopropyl myristate, 0.86 g DuPont Vazo-67, 0.69 g Wako Chemicals V-501, is added to a 35° C. temperature controlled steel jacketed reactor, with mixing at 1000 rpm (4 tip, 2″ diameter, flat mill blade) and a nitrogen blanket applied at 100 cc/min. The oil solution is heated to 70° C. in 45 minutes, held at 75° C. for 45 minutes, and cooled to 50° C. in 75 minutes. This mixture is hereafter referred to as oil solution A.

In a reactor vessel, an aqueous solution is prepared consisting of 300 g of deionized water to which is dispersed in 2.40 grams of Celvol 540 polyvinyl alcohol at 25° C. The mixture is heated to 85° C. and held there for 45 minutes. The solution is cooled to 30° C. 1.03 grams of Wako Chemicals V-501 initiator is added, along with 0.51 grams of a 40% sodium hydroxide solution. The solution is then heated to 50° C., and the solution is maintained at that temperature.

To oil solution A, add 0.19 grams of tert-butyl amino ethyl methacrylate (Sigma Aldrich), 0.19 grams of beta-carboxy ethyl acrylate (Sigma Aldrich), and 15.41 grams of Sartomer CN975 (Sartomer, Inc.). Mix the acrylate monomers into the oil phase for 10 minutes. This mixture is hereafter referred to as oil solution B. Use a Caframo mixer with a 4-blade pitched turbine agitator to achieve the desired oil-in-water emulsion particle size.

Start a nitrogen blanket on top of the aqueous solution in reactor. Start transferring oil solution B into the aqueous solution in the reactor with minimal mixing. Increase the agitation of mixing to 1800-2500 rpm for a period of 60 minutes to emulsify the oil phase into the water solution. After milling is completed, mixing is continued with a 3″ propeller at 350 rpm. The batch is then held at 50° C. for 45 minutes. The temperature is then increased to 75° C. in 30 minutes, held at 75° C. for 4 hours, heated to 95° C. in 30 minutes and held at 95° C. for 6 hours. The batch is then allowed to cool to room temperature.

The resultant microcapsules have a median particle size of 12.6 microns.

### Example 2

### Perfume Microcapsules

An oil solution, consisting of 128.4 g of perfume oil B, 32.1 g isopropyl myristate, 0.86 g DuPont Vazo-67, 0.69 g Wako Chemicals V-501, is added to a 35° C. temperature controlled steel jacketed reactor, with mixing at 1000 rpm (4 tip, 2″ diameter, flat mill blade) and a nitrogen blanket applied at 100 cc/min. The oil solution is heated to 70° C. in 45 minutes, held at 75° C. for 45 minutes, and cooled to 50° C. in 75 minutes. This mixture is hereafter referred to as oil solution A.

In a reactor vessel, an aqueous solution is prepared consisting of 300 g of deionized water to which is dispersed in 2.40 grams of Celvol 540 polyvinyl alcohol at 25° C. The mixture is heated to 85° C. and held there for 45 minutes. The solution is cooled to 30° C. 1.03 grams of Wako Chemicals V-501 initiator is added, along with 0.51 grams of a 40% sodium hydroxide solution. The solution is then heated to 50° C., and the solution is maintained at that temperature.

To oil solution A, add 0.19 grams of tert-butyl amino ethyl methacrylate (Sigma Aldrich), 0.19 grams of beta-carboxy ethyl acrylate (Sigma Aldrich), and 15.41 grams of Sartomer CN975 (Sartomer, Inc.). Mix the acrylate monomers into the oil phase for 10 minutes. This mixture is hereafter referred to as oil solution B. Use a Caframo mixer with a 4-blade pitched turbine agitator to achieve the desired oil-in-water emulsion particle size.

Start a nitrogen blanket on top of the aqueous solution in reactor. Start transferring oil solution B into the aqueous solution in the reactor with minimal mixing. Increase the agitation of mixing to 1800-2500 rpm for a period of 60 minutes to emulsify the oil phase into the water solution. After milling is completed, mixing is continued with a 3″ propeller at 350 rpm. The batch is then held at 50° C. for 45 minutes. The temperature is then increased to 75° C. in 30 minutes, held at 75° C. for 4 hours, heated to 95° C. in 30 minutes and held at 95° C. for 6 hours. The batch is then allowed to cool to room temperature.

The resultant microcapsules have a median particle size of 12.6 microns.

### Examples 3A-3L

### Leave-on Conditioners

### Formula A1-A4

| | Example | Example | Example | Example |
|---|---|---|---|---|
| | A1 | A2 | A3 | A4 |
| Material | (grams) | (grams) | (grams) | (grams) |
| PREMIX | | | | |
| Water | 19.65 | 19.26 | 18.14 | 18.27 |
| Silicone | 0.57 | 1 | 1.5 | 2 |
| Cetyl, Stearyl, Oleyl | 0.59 | 0.75 | 0.85 | 0.65 |
| alcohol | | | | |
| Behenyl | 0.21 | 0.25 | 0.3 | 0.28 |
| Trimethylammonium | | | | |
| chloride BTMAC | | | | |
| Stearamidopropyl | 0.35 | 0.3 | 0.45 | 0.32 |
| Dimethylamine | | | | |
| Preservatives | 0.5 | 0.5 | 0.5 | 0.5 |
| EDTA | 0.22 | 0.22 | 0.22 | 0.22 |
| Panthenyl ethyl ether | 0.31 | 0.25 | 0.35 | 0.28 |
| Hydroxyethyl cellulose | 0.32 | 0.35 | 0.4 | 0.28 |
| Polyethylene glycol PEG | 0.28 | 0.25 | 0.27 | 0.24 |
| 2M | | | | |
| Quaternium-18 | 0.32 | 0.3 | 0.35 | 0.28 |
| Citric acid - anhydrous | 0.22 | 0.22 | 0.22 | 0.22 |
| POST-ADDS | | | | |
| Water | 0.21 | 0.1 | 0.2 | 0.21 |

| | Example | Example | Example | Example | Example | Example |
|---|---|---|---|---|---|---|
| Material | 3A | 3B | 3C | 3D | 3E | 3F |
| Formula | 23.750 g | 23.750 g | 23.750 g | 23.750 g | 23.750 g | 23.750 g |
| A1, A2, | | | | | | |
| A3, or A4 | | | | | | |
| Example 1 | 0 | 0 | 0 | 0.241 g | 0.482 g | 0.803 g |
| Example 2 | 0.285 g | 0.570 g | 0.950 g | 0 | 0 | 0 |
| Water | Q.S. | Q.S. | Q.S. | Q.S. | Q.S. | Q.S. |

| | Example | Example | Example |
|---|---|---|---|
| Material | 3G | 3H | 3I |
| Formula A1, A2, | 7.125 g | 14.250 g | 23.750 g |
| A3, or A4 | | | |
| Example 2 | 0.285 g | 0.570 g | 0.950 g |
| Water | Q.S. | Q.S. | Q.S. |

| Material | Example 3J | Example 3K | Example 3L |
|---|---|---|---|
| Formula A1, A2, A3, | 23.750 g | 23.750 g | 23.750 g |
| or A4 | | | |
| Example 2 | 0.285 g | 0.570 g | 0.950 g |
| Neat Perfume Oil | 0.100 g | 0.100 g | 0.100 g |
| Water | Q.S. | Q.S. | Q.S. |

The microcapsules of Examples 1 & 2 are added to the leave-on treatment Formula A that is preweighed in a 60 ml plastic jar. The composition is then mixed using a Speed Mixer (HAFC 400 DVZ, FlackTek of Landrum, S.C.) at 800 RPM for 1 minute. Examples 3A to 3I are used to prepare hair switches per the Hair Switch Treatment Method and allowed to dry for 4 hours.

Next, the Olfactive Analysis Method is utilized to gather perfume intensity data on the prepared hair switches before and after combing. The hair switches are combed a second time, then the Olfactive Analysis Method is utilized, with the exception that no perfume intensity data is obtained. The hair switches are allowed to age for 24 hours then the same procedure is followed to obtain perfume intensity data.

### Examples 4A-4B

### Leave-on Spray Chassis

| | FORMULA B1-B6 | | | | | |
|---|---|---|---|---|---|---|
| | B1 | B2 | B3 | B4 | B5 | B6 |
| RM name | wt % | wt % | wt % | wt % | wt % | wt % |
| Water | 97.28% | 97.73% | 96.99% | 97.78% | 97.23% | 98.00% |
| Benzyl Alcohol | 0.40% | 0.40% | 0.40% | 0.40% | 0.40% | 0.40% |
| Phenoxyethanol and | 1.00% | 1.00% | 1.00% | 0.50% | 1.00% | 1.00% |
| Ethylhexylglycerin | | | | | | |
| (Euxyl PE 9010) | | | | | | |
| Polyacrylamide & | 0.82% | 0.00% | 1.11% | 0.82% | 0.00% | 0.00% |
| C13-14 Isoparaffin Laureth-7 | | | | | | |
| Polyacrylate | 0.00% | 0.37% | 0.00% | 0.00% | 0.37% | 0.00% |
| Crosspolymer-6 | | | | | | |
| Acrylates/Vinyl | 0.00% | 0.00% | 0.00% | 0.00% | 0.00% | 0.10% |
| Isodecanoate Crosspolymer | | | | | | |
| 10000 cps | 0.50% | 0.50% | 0.50% | 0.50% | 1.00% | 0.50% |
| Amodimethicone | | | | | | |
| Total | 100.00% | 100.00% | 100.00% | 100.00% | 100.00% | 100.00% |

| Material | Example 4A | Example 4B |
|---|---|---|
| Formula B1, B2, B3, B4, B5, | 23.750 g | 23.750 g |
| or B6 | | |
| Example 2 | 0.950 g | 0.285 g |
| Water | Q.S. | Q.S. |

### Further Examples/Combinations

A. A method for treating hair, the method comprising:
   (i) providing a hair conditioner composition, the composition comprising a solute and a carrier, said solute comprising a conditioning agent and a plurality of microcapsules, wherein said microcapsules comprise an encapsulated perfume oil, wherein the weight ratio of the encapsulated perfume oil to the solute is greater than 0.02; and
   (ii) applying from 0.01 grams to 3.4 gram per gram of hair or less per use.
B. The method of paragraph A, wherein the hair conditioning composition comprises from 0.01% to 45%, by weight, of the microcapsules.
C. The method of paragraph A or B, wherein the weight ratio of the encapsulated perfume oil to solute is from 0.02 to 0.7.
D. The method of paragraphs A-C, wherein the weight ratio of the encapsulated perfume oil to solute is from 0.1 to 0.5.
E. The method of paragraphs A-D, wherein 0.01 grams to 0.1 gram per gram of hair or less is applied per use.
F. The method of paragraphs A-E, wherein the microcapsules further comprise a shell material selected from the group consisting of polyacrylates, polyethylenes, polyamides, polystyrenes, polyisoprenes, polycarbonates, polyesters, polyureas, polyurethanes, polyolefins, polysaccharides, epoxy resins, vinyl polymers, urea cross-linked with formaldehyde or gluteraldehyde, melamine cross-linked with formaldehyde; gelatin-polyphosphate coacervates optionally cross-linked with gluteraldehyde; gelatin-gum Arabic coacervates; cross-linked silicone fluids; polyamine reacted with polyisocyanates; acrylate monomers polymerized via free radical polymerization, as well as naturally occurring materials selected from the group consisting of silk, wool, gelatine, cellulose, proteins, and combinations thereof.
G. The method of paragraphs A-F, wherein the microcapsules further comprise a partitioning modifier selected from the group consisting of isopropyl myristate, mono-, di-, and tri-esters of C₄-C₂₄ fatty acids, castor oil, mineral oil, soybean oil, hexadecanoic acid, methyl ester isododecane, isoparaffin oil, polydimethylsiloxane, brominated vegetable oil, and combinations thereof.
H. The method of paragraphs A-G, wherein the microcapsules further comprise isopropryl myristate.
I. The method of paragraphs A-H, wherein the hair conditioning composition comprises from 0.01% to 12%, by weight, of the conditioning agent.
J. The method of paragraphs A-I, further comprising a rheology modifier.
K. The method of paragraphs A-J, wherein the hair conditioning composition further comprises from 0.01% to 3%, by weight, of a rheology modifier
L. The method of paragraphs A-K, further comprising a non-encapsulated perfume oil.
M. The method of paragraphs A-L, further comprising a silicone.
N. The method of paragraphs A-M, wherein the microcapsules comprise a shell, said shell being a polyacrylate shell.
O. The method of paragraphs A-N, further comprising a silicone.

It is further noted that terms like “preferably,” “usually”, “generally,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is additionally noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A method for treating hair, the method comprising:
(iii) providing a hair conditioner composition, the composition comprising a solute and a carrier, said solute comprising a conditioning agent and a plurality of microcapsules, wherein said microcapsules comprise an encapsulated perfume oil, wherein the weight ratio of the encapsulated perfume oil to the solute is greater than about 0.02; and
(iv) applying from about 0.01 grams to about 3.4 gram per gram of hair or less per use.

2) The method of claim 1, wherein the hair conditioning composition comprises from 0.01% to 45%, by weight, of the microcapsules.

3) The method of claim 1, wherein the weight ratio of the encapsulated perfume oil to solute is from about 0.02 to about 0.7.

4) The method of claim 1, wherein the weight ratio of the encapsulated perfume oil to solute is from about 0.1 to about 0.5.

5) The method of claim 1, wherein about 0.01 grams to about 0.1 gram per gram of hair or less is applied per use.

6) The method of claim 1, wherein the microcapsules further comprise a shell material selected from the group consisting of polyacrylates, polyethylenes, polyamides, polystyrenes, polyisoprenes, polycarbonates, polyesters, polyureas, polyurethanes, polyolefins, polysaccharides, epoxy resins, vinyl polymers, urea cross-linked with formaldehyde or gluteraldehyde, melamine cross-linked with formaldehyde; gelatin-polyphosphate coacervates optionally cross-linked with gluteraldehyde; gelatin-gum Arabic coacervates; cross-linked silicone fluids; polyamine reacted with polyisocyanates; acrylate monomers polymerized via free radical polymerization, as well as naturally occurring materials selected from the group consisting of silk, wool, gelatine, cellulose, proteins, and combinations thereof.

7) The method of claim 1, wherein the microcapsules further comprise a partitioning modifier selected from the group consisting of isopropyl myristate, mono-, di-, and tri-esters of C₄-C₂₄ fatty acids, castor oil, mineral oil, soybean oil, hexadecanoic acid, methyl ester isododecane, isoparaffin oil, polydimethylsiloxane, brominated vegetable oil, and combinations thereof.

8) The method of claim 1, wherein the microcapsules further comprise isopropryl myristate.

9) The method of claim 1, wherein the hair conditioning composition comprises from about 0.01% to about 12%, by weight, of the conditioning agent.

10) The method of claim 1, further comprising a rheology modifier.

11) The method of claim 1, wherein the hair conditioning composition further comprises from about 0.01% to about 3%, by weight, of a rheology modifier

12) The method of claim 1, further comprising a non-encapsulated perfume oil.

13) The method of claim 1, further comprising a silicone.

14) The method of claim 1, wherein the microcapsules comprise a shell, said shell being a polyacrylate shell.

15) The method of claim 1, further comprising a silicone.
